(19) United States
(12) Patent Application Publication
(10) Pub. No.: US 2018/0372753 A1
(43) Pub. Date: Dec. 27, 2018

(54) Method for the Determination of Biomolecule Turnover Rates

(71) Applicant: The Regents of the University of California, Oakland, CA (US)

(72) Inventors: Peipei PING, Redondo Beach, CA (US); Tae-Young KIM, Gwangju (KR); Ding WANG, Van Nuys, CA (US); Allen KIM, Tujunga, CA (US); Edward LAU, Van Nuys, CA (US); David A. LIEM, Los Angeles, CA (US); Pui Yu LAM, Van Nuys, CA (US); Mario DENG, Venice, CA (US)

(73) Assignee: The Regents of the University of California, Oakland, CA (US)

(21) Appl. No.: 15/927,772

(22) Filed: Mar. 21, 2018

Related U.S. Application Data
(63) Continuation of application No. 14/759,149, filed on Jul. 2, 2015, now abandoned.
(60) Provisional application No. 61/748,971, filed on Jan. 4, 2013.
(60) Provisional application No. 61/839,837, filed on Jun. 26, 2013.

(51) Int. Cl. G01N 33/68; G01N 33/58; G01N 33/60; G01N 30/72; G01N 27/447; G01N 30/88

(52) U.S. Cl. CPC G01N 33/6848; G01N 33/58; G01N 33/60; Y10T 436/24; G01N 2458/15; G01N 30/72; G01N 27/447; G01N 2030/8813; Y10T 436/143333

(57) ABSTRACT

Disclosed is a method for determining the turnover rate of biomolecules in a subject, which include administering to the subject, ²H₂O in an amount sufficient to label biomolecules in the subject with ²H. Samples are collected from the subject at one or more time points and isotopomers are detected for the labeled biomolecules in the samples. The fractional abundance is determined for the isotopomers of the biomolecules in the samples and the biomolecule turnover rates of the one or more labeled biomolecules is determined based on the fractional abundance of the isotopomers. A computer-implemented method is also disclosed for determining the turnover rate of one or more biomolecules in subject. In certain other embodiments, a system for determining protein turnover rates in a subject is also provided. Also provided in certain embodiments is a computer program product for determining protein turnover rates in a subject.

### CROSS-REFERENCE TO RELATED APPLICATIONS

This application is a continuation of U.S. application Ser. No. 14/759,149, filed Jul. 2, 2015, which is a 371 National Phase Application of PCT/US2014/010176, filed Jan. 3, 2014, which claims the benefit of U.S. Provisional Application No. 61/748,971, filed Jan. 4, 2013 and U.S. Provisional Application No. 61/839,837, filed Jun. 26, 2013. These applications are incorporated herein by reference in their entirety.

### STATEMENT OF GOVERNMENT INTEREST

This invention was made with Government support under Grant Nos. HHSN268201000035C, HL063901, awarded by the National Institutes of Health. The Government has certain rights in the invention.

### FIELD

This disclosure relates to the field of biomedical and therapeutic technology. More specifically, this disclosure relates to analytical and computational methods of determining biomolecule turnover rates using heavy water labeling.

### BACKGROUND

The maintenance of biological functions requires a delicate balance of its continuous protein synthesis and degradation, i.e., protein turnover. For example, inadequate protein quality control is often observed in mitochondrial dysfunctions in disorders including neurodegenerating phenotypes, cardiovascular diseases, and aging. It is postulated that the failure to contain or replenish the proteins damaged by reactive oxygen species directly underlies many pathological phenotypes. The development of effective treatments for these diseases therefore relies on understanding the molecular basis of protein dynamics. An outstanding question is how the processes of proteome dynamics are regulated in different systems, and how their perturbations could progress to pathological remodeling. Thus far, quantitative proteomics efforts are predominated by steady-state measurements, which often provide fragmentary snapshots of the proteome that are difficult to comprehend in the context of other cellular events. Therefore methods that enable the characterization of biomolecular kinetics are essential to further the understanding of these biological processes and their roles in disease.

### SUMMARY

Disclosed is a method for determining the turnover rate of at least one or more biomolecules in a subject. In some embodiments, the methods include administering to the subject, ²H₂O in an amount sufficient to label the at least one or more biomolecules in the subject with ²H. Samples are collected from the subject at one or more time points and one or more isotopomers or detected (for example by mass spectral analysis) of the at least one or more labeled biomolecules in the samples. The fractional abundance is determined for the one or more isotopomers of the at least one labeled biomolecule in the samples and the biomolecule turnover rates of the one or more labeled biomolecules is determined based on the fractional abundance of the one or more isotopomers.

A computer-implemented method is also disclosed for determining the turnover rate of one or more biomolecules in subject. In some embodiments, the method includes: receiving, by one or more computing devices, mass spectra data from samples collected from a subject at one or more time points, wherein the one or more biomolecules in the subject have been labeled with ²H; receiving, by the one or more computing devices, biomolecule identification data; parsing, by the one or more computing devices, the mass spectra data and the biomolecule identification data; assigning, by the one or more computing devices, mass spectral data to biomolecular identification data to identify peaks in the mass spectral data; integrating, by the one or more computing devices, peaks in the mass spectral data to determine fractional abundance of one or more isotopomers of ²H labeled biomolecules in the samples; receiving, by the one or more computing devices, enrichment rate and level data; and fitting, by the one or more computing devices, the fractional abundance of the one or more isotopomers of 2H labeled biomolecules in the samples to a equation describing labeled biomolecule turn over to determine the molecular turnover rates of biomolecules in the subject.

In certain other embodiments, a system for determining protein turnover rates in a subject is also provided. Also provided in certain embodiments is a computer program product for determining protein turnover rates in a subject.

The foregoing and other objects, features, and advantages of the invention will become more apparent from the following detailed description, which proceeds with reference to the accompanying figures.

### BRIEF DESCRIPTION OF THE DRAWINGS

FIG. 1 shows metabolic labeling of mice using heavy water. (A) a schematic of ²H₂O labeling of mouse and sample collection. Mice were labeled by heavy water via a combined IP injection of 99.9% ²H₂O/saline and 8% ²H₂O drinking. Samples were collected at multiple time points. (B)²H₂O labeling introduces ²H-labeled amino acids into the precursor pool for protein synthesis. (C) molar percent enrichment of ²H in mouse serum during ²H₂O feeding was measured by GC-MS at 13 time points. Enrichment reached 3.5% within 12 h after two IP injections of 99.9% ²H₂O/saline and plateaued at ˜4.3% throughout the labeling period with 8% ²H₂O feeding.

FIG. 2 is a set of graphs showing the extraction of protein turnover rates from the temporal profile of mass isotopomer distribution. (A) the profile of the relative abundances for mass isotopomers of as a function of labeling time. ²H₂O labeling for 90 d resulted in a change in the relative abundances of mass isotopomers. The values for A₀(0)=0.52, A₀(∞)=0.18, and k=0.066 d⁻¹ for m₀ of the peptide were obtained by fitting to an exponential curve (R²=0.99), then transformed into fractional synthesis, f(t), with the following equation: f(t)={A(t)−A(0)}/{A(∞)−A(0)}. (B) protein turnover rate was determined by fitting the fractional syntheses of mass isotopomers of a protein throughout the labeling period into an exponential curve. The turnover rates for this protein in the heart and the liver are 0.065±0.004 d⁻¹ (R²=0.98) and 0.205±0.028 d⁻¹ (R²=0.95), respectively.

FIG. 3 is a schematic of the analyses of turnover rates of mitochondrial proteins identified in both heart and liver. The cardiac k_{deg} values are plotted in ascending order on a logarithmic scale and paired with the corresponding hepatic k_{deg} values from the same protein. Among the 242 proteins analyzed in both organs, only 3 had smaller turnover rates in the liver than in the heart. Error bars represent SEM.

FIG. 4 is a set of graphs of the distributions of protein turnover rates and their correlations with functions. (A) histograms of protein turnover rates in heart and liver mitochondria. Proteins in the heart have slower turnover rates than those in the liver (median k=0.042 d⁻¹ vs. 0.163 d⁻¹). (B) the measured turnover rates of murine mitochondrial proteins against their Gene Ontology categories (GO). Box: interquartile range and median; whiskers: data range up to 1.5 interquartile ranges. The numbers of analyzed proteins in the category are parenthesized.

FIG. 5 is a set of plots showing the factors affecting mitochondrial protein turnover. (A) protein turnover rates in the heart and the liver were significantly correlated (Spearman's ρ=0.50). (B) PEST motifs and, (C) intrinsic protein sequence disorders were not indicative of protein turnover rates. (D) comparison between sub-mitochondrial locations revealed that median turnover is higher in the outer membrane than in the inner membrane. The solid and dotted lines in B, C, and D denote the median and the interquartile range, respectively.

FIG. 6 shows the correlation between protein turnover rates and biophysical parameters. (A) a weak inverse correlation was observed between protein turnover rate and relative protein abundance (heart: ρ=−0.46, P<2.2×10⁻¹⁶ and liver: ρ=−0.19, P=7.95×10⁻³), suggesting abundant proteins are turned over more slowly in general. The relative abundance of a protein was determined by the summation of total chromatographic areas of the constituent peptide ion peaks divided by the areas of all identified peptide ions in the experimental dataset using Progenesis LC-MS (Nonlinear Dynamics). By contrast, no significant correlations were observed in either tissue between protein turnover rates and their molecular weights, (B) or their isoelectric points, (C) or their hydrophobicities, (D).

FIG. 7 is a histogram of the standard errors in the rate constants for cardiac mitochondria proteins. The standard errors (σk) in the rate constants for cardiac mitochondrial protein turnover were calculated using both the Monte Carlo and the Non-linear curve fitting methods. The distributions of the standard errors are not significantly different, although the Monte Carlo method is more conservative in the estimated errors.

FIG. 8 is a plot of the mitochondrial protein turnover rates in the heart and the liver. The protein turnover rates (k) of all analyzed murine mitochondrial proteins in the liver and in the heart are displayed on linear, non-logarithmic scale based on protein functional categories. The median turnover rates in the heart and the liver were 0.04 and 0.163 d⁻¹, respectively. The number in the parenthesis represents the total number of proteins belonging to a functional category. Cardiac and hepatic mitochondrial proteins are indicated.

FIG. 9 is a depiction of fitting the area under the curve of a mass spec peak.

FIG. 10 is a block diagram of depicting a method for determining the turnover rate of a biomolecule in a subject, in accordance with certain example embodiments.

FIG. 11 is a block diagram of depicting a method for determining the turnover rate of a biomolecule in a subject, in accordance with certain example embodiments.

FIG. 12 is a block diagram of depicting a method for integration of a peak in a mass spectrum, in accordance with certain example embodiments.

FIG. 13 is a block diagram of depicting a method for curve fitting integration data, in accordance with certain example embodiments.

FIG. 14 is a block diagram of depicting a method for comparing results, in accordance with certain example embodiments.

FIG. 15 is a block diagram of depicting a method for generating tables and graphs, in accordance with certain example embodiments.

FIG. 16A is a set of graphs showing how ²H₂O (heavy water) labeling in human differs from that in the mouse. In the mouse, constant label enrichment can be easily achieved through a priming injection of heavy water to bring total enrichment to the desired level. In contrast, small boluses of heavy water are given to the human subjects for gradual intake, thus label enrichment rises gradually before reaching the target level. The observed pattern of isotope appearance in the proteins therefore follows a sigmoidal shape as in the nonlinear function described below, which cannot be modeled using a simple exponential decay equation.

FIG. 16B is a schematic representation of a typical heavy water labeling study to study protein turnover in human. The human subjects were instructed to intake 4 boluses of 0.51-mL·kg-1 (body mass) sterile 70% molar ratio heavy water per day for the first 7 days; and 2 boluses of 0.56-mL·kg-1 sterile 70% molar ratio heavy water per day for the next 7 days. Blood samples were collected over a time course at 10 to 15 time points and analyzed by mass spectrometry. The data were then processed by ProTurn using nonlinear modeling to deduce the protein turnover rates.

FIG. 17 shows the in vivo protein turnover rates (and by extension, protein half-life) of 183 human blood proteins that were measured in at least 3 individual subjects. The x-axis represents the index of the proteins analyzed. The y-axis represents the log 10 value of turnover rate (% replaced per day) of the protein, which also gives its half-life (ln(2)/turnover rate). These data were acquired using the methods described herein. In total, four subjects were labeled with heavy water for 2 weeks, and blood was drawn at 10 to 15 time points to measure both the subjects' heavy water enrichment using GC-MS and the protein isotope incorporation using LC with high-resolution MS. The GC-MS data were modeled using a first-order exponential decay function to yield the rate constant and plateau level of heavy water enrichment, which were then fed into the nonlinear function to deduce the protein turnover rate from the LC-MS data using computational optimization in ProTurn. These data demonstrate the utility of the method for measuring protein half-life. The method will be applicable to comparing protein half-life among individuals of particular phenotypes and also in the same individuals before and after the onset of diseases, as a means to identify quantitative molecular markers of disease progression, susceptibility and/or treatment response. In total, the in vivo turnover rates of over 500 proteins have been an acquired, which represents the biggest human protein turnover rate dataset to-date.

FIGS. 18A-18C is a set of graphs showing that the disclosed method is applicable to deducing protein turnover rate from protein samples taken at just a single time point. This is because when using a nonlinear modeling method, the initial and plateau values of protein label incorporation can be estimated using the exponential decay curve of heavy water enrichment plus protein sequence information. FIG. 18A is a graph of a computer simulation of how the kinetic curves will look like under different turnover rates. It can be seen that protein isotope incorporation data taken at a single time point would be sufficient to differentiate the kinetic curves and deduce protein half-life without time course information. Note the sigmoidal shape of the curve that is a telltale sign of the disclosed nonlinear dual-rate-constants model. FIG. 18B is a graph that shows actual experimental data from protein samples taken from a human subject at day 8 after the commencement of heavy water labeling, and the protein half-life calculated from the data. FIG. 18C is a graph that shows a comparison of the large-scale turnover rate information acquired by this one-point method with the information acquired from the more conventional time-course method. Such application is one of the distinguishing features of the algorithm.

FIG. 19 shows the increased protein turnover (or decreased half-life) of almost all proteins in the glycolysis pathways during cardiac remodeling induced by chronic administration of isoproterenol, a cardiac hypertrophy stimulus in mouse models. It also shows the difference in protein turnover between glycolysis and fatty acid oxidation proteins in the early failing heart, demonstrating that the kinetic responses are specific and correspond to protein pathways.

FIG. 20 is a graph showing that ProTurn allows both protein turnover and abundance to be quantified from a heavy water labeling experiment. The figure shows that the changes in protein turnover (or half-life) and changes in protein abundance following the onset of early-stage heart failure in mice are in fact poorly correlated, i.e., protein half-life is effectively an independent parameter. These data highlight that there is added value in performing protein turnover rate measurements using the method described, and that such experiments have the potential to discover additional molecular changes in disease models over protein abundance measurement alone.

FIG. 21 is a block diagram depicting a computing machine and a module, in accordance with certain example embodiments.

FIG. 22 is a table showing protein turnover rates.

### DETAILED DESCRIPTION

### I. Explanation of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of specific terms are provided:

Administering: Administering refers to the introduction of a composition into a subject by a chosen route, for example the administration of heavy water to a subject, such as a human subject.

Biological sample: Any solid or fluid sample obtained from, excreted by or secreted by any organism, including without limitation, multicellular organisms (animals, including samples from a healthy or apparently healthy human subject or a human patient affected by a condition or disease to be diagnosed or investigated). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease such as a rheumatoid arthritis, osteoarthritis, gout or septic arthritis). A biological sample can also be a sample obtained from any organ or tissue or can comprise a cell (whether a primary cell or cultured cell) or medium conditioned by any cell, tissue or organ or subcellular fraction, such as a mitochondria. In some examples, a biological sample is an artificial sample.

Chromatography: The process of separating a mixture. It involves passing a mixture through a stationary phase, which separates molecules of interest from other molecules in the mixture and allows it to be isolated. Examples of methods of chromatographic separation include capillary-action chromatography such as paper chromatography, thin layer chromatography (TLC), column chromatography, fast protein liquid chromatography (FPLC), nanoflow reversed-phase liquid chromatography, ion-exchange chromatography, gel chromatography such as gel filtration chromatography, size exclusion chromatography, affinity chromatography, high performance liquid chromatography (HPLC), and reversed-phase high performance liquid chromatography (RP-HPLC) amongst others.

Corresponding: The term “corresponding” is a relative term indicating similarity in position, purpose or structure. In some embodiments, mass spectral signals in a mass spectrum that are due to corresponding peptides of identical structure but differing masses are “corresponding” mass spectral signals. A mass spectral signal due to a particular peptide is also referred to as a signal corresponding to the peptide.

Fragment peptide: A peptide that is derived from the full length protein, through processes including fragmentation, enzymatic proteolysis, or chemical hydrolysis. Such proteolytic peptides include peptides produced by treatment of a protein with one or more endoproteases such as trypsin, chymotrypsin, endoprotease ArgC, endoprotease AspN, endoprotease GluC, and endoprotease LysC, as well as peptides produced by cleavage using chemical agents, such as cyanogen bromide, and hydrochloric acid. Fragment peptides can be used as mass identifiers for the presence of a protein in a sample, such as a sample obtained from a subject.

Heavy water or deuterium oxide (²H₂O or D₂O): A form of water that contains the hydrogen isotope deuterium.

Isolated: An “isolated” biological component (such as a nucleic acid, peptide, protein, lipid, or metabolite) has been substantially separated, produced apart from, or purified away from other biological components in the cell of the organism in which the component naturally occurs or is transgenically expressed, that is, other chromosomal and extrachromosomal DNA and RNA, proteins, lipids, and metabolites. Nucleic acids, peptides, proteins, lipids and metabolites which have been “isolated” thus include nucleic acids, peptides, proteins, lipids, and metabolites purified by standard or non-standard purification methods. The term also embraces nucleic acids, peptides, proteins, lipids, and metabolites prepared by recombinant expression in a host cell as well as chemically synthesized peptides, lipids, metabolites, and nucleic acids.

Isotopic analog or isotopomers: A molecule that differs from another molecule in the relative isotopic abundance of an atom it contains. For example, peptide sequences containing identical sequences of amino acids, but differing in the isotopic abundance of an atom, are isotopic analogs of each other, for example the abundance of ²H and ¹H.

Isotopically-labeled or labeled: “Isotopically-labeled” or “labeled” refer to a molecule that includes one or more isotopes, either stable or radioactive, heavy or light, in a greater-than-natural abundance. For example, ²H, ¹³C, ¹⁵N, and ¹⁸O are heavy isotopes of elements commonly found in biomolecules; whereas, ¹²³I and ¹²⁵I are light isotopes of natural ¹²⁷I.

Mass spectrometry: Mass spectrometry is a method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography.

Peptide/Protein/Polypeptide: All of these terms refer to a polymer of amino acids and/or amino acid analogs that are joined by peptide bonds or peptide bond mimetics. The twenty naturally-occurring amino acids and their single-letter and thrce-letter designations are as follows:

| Amino Acid | Single-letter Symbol | Three-letter Symbol |
|---|---|---|
| Alanine | A | Ala |
| Cysteine | C | Cys |
| Aspartic Acid | D | Asp |
| Glutamic acid | E | Glu |
| Phenylalanine | F | Phe |
| Glycine | G | Gly |
| Histidine | H | His |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |

Predictable mass difference: A predictable mass difference is a difference in the molecular mass of two molecules or ions (such as two peptides, peptide ions) that can be calculated from the molecular formulas and isotopic contents of the two molecules or ions. Although predictable mass differences exist between molecules or ions of differing molecular formulas, they also can exist between two molecules or ions that have the same molecular formula but Include different isotopes of their constituent atoms. A predictable mass difference is present between two molecules or ions of the same formula when a known number of atoms of one or more type in one molecule or ion are replaced by lighter or heavier isotopes of those atoms in the other molecule or ion. For example, replacement of a ¹H atom with a ²H (or vice versa) provides a predictable mass difference of about 1 amu. Such differences between the masses of particular atoms in two different molecules or ions are summed over all of the atoms in the two molecules or ions to provide a predictable mass difference between the two molecules or ions.

Standard: A standard is a substance or solution of a substance of known amount, purity or concentration. A standard can be compared (such as by spectrometric, chromatographic, or spectrophotometric analysis) to an unknown sample (of the same or similar substance) to determine the presence of the substance in the sample and/or determine the amount, purity or concentration of the unknown sample. In one embodiment a standard is a peptide standard. An internal standard is a compound that is added in a known amount to a sample prior to sample preparation and/or analysis and serves as a reference for calculating the concentrations of the components of the sample. Isotopically-labeled peptides are particularly useful as internal standards for peptide analysis since the chemical properties of the labeled peptide standards are almost identical to their non-labeled counterparts. Thus, during chemical sample preparation steps (such as chromatography, for example, HPLC) any loss of the non-labeled peptides is reflected in a similar loss of the labeled peptides.

Subject: Any living or once living organisms or sub-fractions thereof a category that includes both human, non-human mammals, drosophila, zebrafish, yeast, bacteria, and cells, whether primary, cultured, natural, metabolically modified, chemically engineered, or genetically engineered.

### II. Description of Several Embodiments

### A Introduction

Proteome turnover dynamics provides an important description of cellular homeostasis on systems levels, and contributes to the discrepancies between transcriptome and proteome expressions. There is a growing interest in measuring protein dynamics in vivo, spurred by the promises of novel kinetics-based diagnostic protein biomarkers and mechanistic insights into cellular physiology. Much has been learned on protein stability and degradation from large-scale in vitro experiments, e.g., using dynamic SILAC labeling. It is thought, however, that protein turnover in cultured cells does not fully recapitulate the additional physiological regulations that occur in multicellular organisms.

As disclosed herein, the inventors have demonstrated that ²H₂O labeling is a viable method for measuring protein turnover in whole organisms. Heavy water (²H₂O) labeling offers several advantages with respect to safety, labeling kinetics, and cost. First, ²H₂O administration to animals and humans at low enrichment levels is safe for months or even years. Second, maintaining constant ²H enrichment levels in body water following the initial intake of ²H₂O is easily achieved, since administrated ²H₂O rapidly equilibrates over all tissues but exits the body slowly (e.g., through body fluid loss). Third, ²H₂O labeling is cost-effective compared with other stable isotope labeling methods. Importantly, ²H₂O intake induces universal ²H incorporation into biomolecules. Systematic insights into protein turnover in vivo could therefore be correlated to that of nucleic acids, carbohydrates, or lipids, enabling broad applications for this technology in studying biological systems, including human. Ingested ²H₂O quickly equilibrates with amino acids to provide a ²H tracer for protein synthesis. Newly synthesized proteins containing ²H-labeled amino acids can then be distinguishable by evolutions in peptide mass isotopomer distribution, the rate of which reflects the synthesis rate of the protein.

One problem with ²H₂O-labeling is that, unlike readily analyzable SILAC data, no software is available to automatically deconvolute ²H₂O-labeled isotopomer distribution data into protein synthesis rate information. Furthermore, measuring proteome dynamics in human is currently difficult. The slow precursor enrichment in human labeling experiments means that a protein molecule synthesized immediately after labeling commences would contain fewer ²H than one that is synthesized later. Thus corrections to the isotopomer distribution of each data point are required in order to deduce the fraction of newly synthesized peptides. Together, these factors contribute as barriers to proteome dynamics studies.

As disclosed herein, the inventors have produced a workflow for measuring protein turnover rates in a subject. The workflow applies in a large scale to drosophila, mice, and humans, cells (e.g., primary cell cultures, transformed cell lines, induced pluripotent stem cells, embryonic stem cells, induced differentiated cells, etc.), and the like. This method can be applied to study the kinetics of other biomolecules including nucleic acids, lipids and metabolites. It can be applied to any animal, cell or part thereof. In some embodiments, animal subjects are fed heavy water and sacrificed at different time points. In humans, saliva, blood, or urine is collected at the different time points. Although this approach applies to proteins, lipids, nucleic acids, the examples given below use protein turnover in the human plasma, human tissues, mouse heart, neonatal rat ventricular myocytes, and adult drosophila to introduce the concept.

Herein described is a novel strategy to determine protein turnover rates on a proteomic scale using ²H₂O labeling. By computing the parameters needed to deduce fractional protein synthesis using software the inventors developed, they were able to obtain protein half-life data without relying on the asymptotic isotopic abundance of peptide ions. Alternatively, this approach can be used to model protein turnover behavior in variable isotope enrichment scenarios. This approach also has the unique benefits of automating all steps of isotopomer quantification and post-collection data analysis, and does not require knowledge of the exact precursor enrichment or labeling sites of peptides. Diverse kinetics from 458 liver and heart mitochondrial proteins was observed that inform essential characteristics of mitochondrial dynamics and intra-genomic differences between the two organs. The turnover rates of 2,964 heart and blood proteins were measured in a mouse heart disease model that suggest widespread kinetic changes in multiple cellular pathways.

Software and technologies as disclosed herein can advance the understanding of the dynamics of lipids, metabolites, proteins, and nucleotides, including the status of protein complex assembly, metabolism and dietary requirements, temporal progressions of phenotypes, etc. Biomolecular turnover rates can be used as biomarkers for disease prevention, prognosis, diagnosis and therapeutic guidances. The clinical utility includes: tracing the effects on biomolecular kinetics before, during, and after treatment of diseases; and monitoring the current health state of patients, which is contributed by genetic predisposition and environmental factors, may be obtained via the patients' dynamic profiles of protein, nucleotide, metabolite, and lipid, given by measuring their turnover rates. A comprehensive measurement of biomolecular turnover rate may provide unique biological signatures or biomarkers to customize healthcare and personalize medicine for patients.

With respect to isotopomers, relative abundance and fractional abundance are often used interchangeably, but may be construed to have a technical distinction: whereas fractional abundance of an isotopomer is the portion of its intensity with respect to the summed intensity of all isotopomers in the peptide envelope, relative abundance of an isotopomer can mean its intensity divided by the intensity of the highest isotopomer or by the value of the arbitrary scale being employed. As used herein, fractional abundance and relative abundance are used to mean the fractional abundance, and relative abundance is used interchangeably with fractional abundance.

### B. Methods

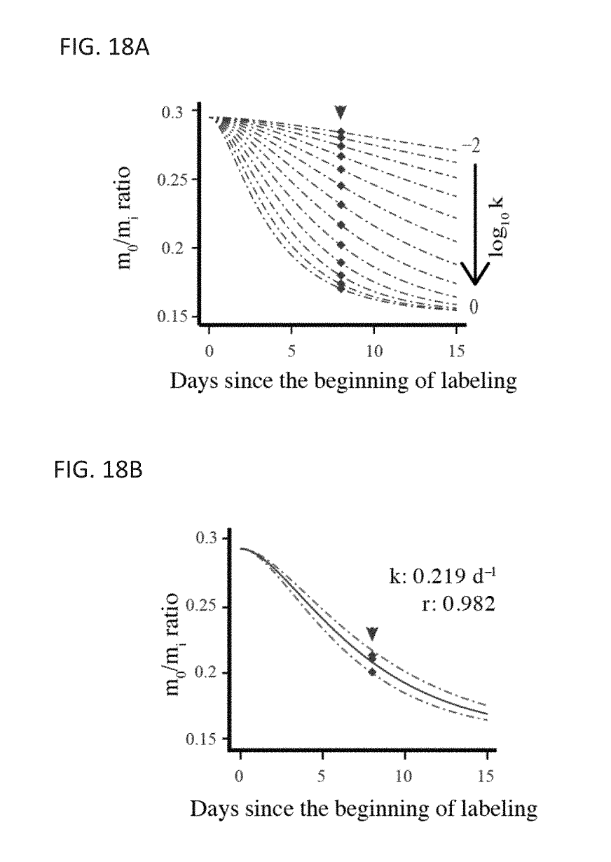

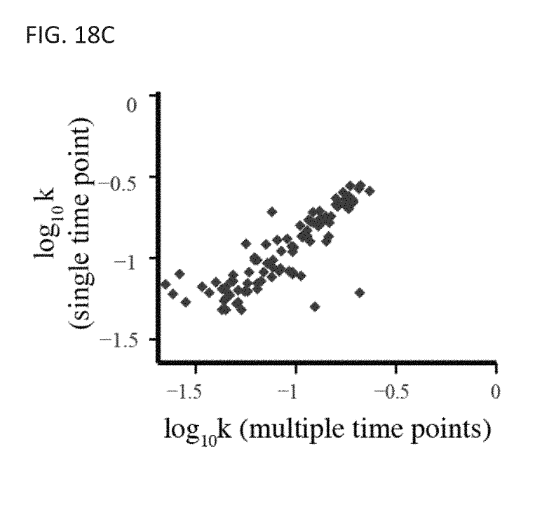

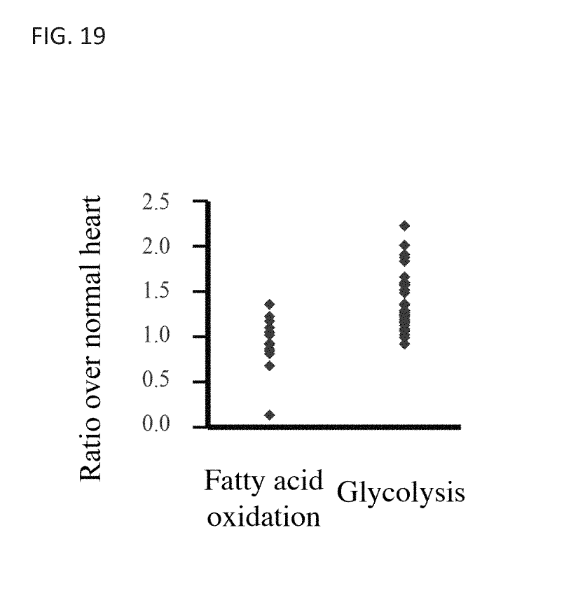

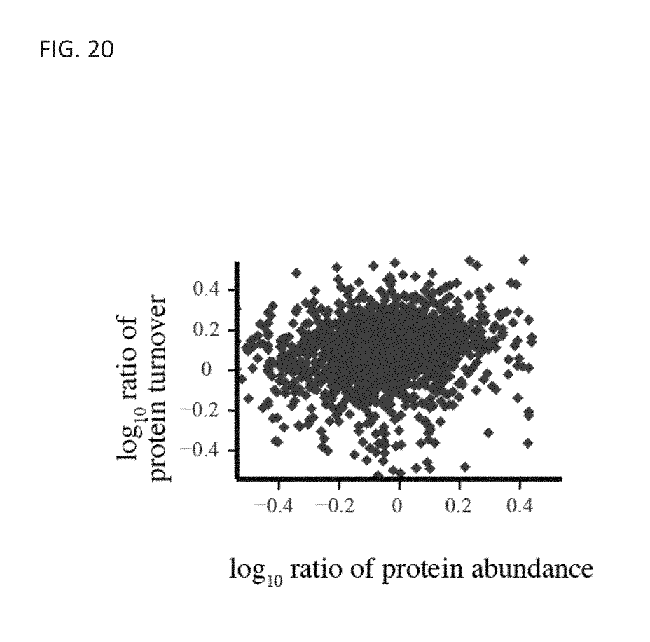

Disclosed herein is a method for determining the turnover rates of at least one or more biomolecules (such as protein, nucleic acids, lipids, glycans, carbohydrates, small molecule metabolites or any other biological material that can be synthesized and can be labeled with ²H for example by incorporation by ²H₂O metabolism) in a subject (such as an organelle, a cell, or an organism), for example a computer implemented method. In the disclosed methods, the subject is administered an amount of ²H₂O, for example in an effective amount, such as in an amount sufficient to label the at least one or more biomolecules in the subject with ²H, such as described herein. Samples are collected from the subject at one or more time points, for example after the administration of the ²H₂O has been discontinued or while the administration of the ²H₂O is continuing. As disclosed herein (see FIGS. 18A-18C and accompanying text), only a single time point is needed to determine biomolecule turn over rates, although a greater number can be used. In some examples a sample is collected from the subject at at least 1 time point, such as at least 1, 2, 3, 4, 5, 6, 7, 8, 9, 10, 11, 12, 13, 14, 15, 16, 17, 18, 19, 20, 25, 30, 40, 50, 75, 100 or more time points, for example between 1 and 10, 3 and 7, 5 and 25, 5 and 13, 7 and 50 and the like. In some examples, less than 100 time points are collected. In some examples, only a singular time point is collected. The samples are analyzed to detect at least one or more labeled biomolecules, for example using mass spectrometry. Although particular relevance is given to the use of mass spectrometry for the detection of labeled biomolecules, any method can be used to detect such biomolecules. Typically in mass spectral analysis a fragment of the biomolecule which can be used to determine the relative abundance of the labeled biomolecule before fragmentation. Thus in this disclosure, the fractional and/or relative abundance of a biomolecule can be used interchangeably with fragments of such biomolecules. In the disclosed methods, the fractional abundance is determined for one or more isotopomers of the at least one labeled biomolecule in the samples at one or more time points. Using the fractional abundances of the one or more isotopomers, the biomolecule turnover rate is determined for the one or more labeled biomolecules, thereby determining the molecular turnover rates of biomolecules in the subject.

In some examples, the samples are subjected to sample pre-processing, for example to purify biomolecules of interest, and/or fragment biomolecules of interest, for example for mass spectral analysis. In some examples, sample pre-processing comprises one or more of gel electrophoresis, liquid chromatography, gas chromatography, capillary electrophoresis, capillary gel electrophoresis, isoelectric focusing chromatography, paper chromatography, thin-layer chromatography; nano-flow chromatography, micro-flow chromatography, high-flow-rate chromatography, reversed-phase chromatography, normal-phase chromatography, hydrophilic-interaction chromatography, ion exchange chromatography, porous graphitic chromatography, size-exclusion chromatography, affinity-based, chromatography, chip-based microfluidics, high-performance liquid chromatography, ultra-high-pressure liquid chromatography or flow-pressure liquid chromatography. In some embodiments, samples can be subjected to 1-dimensional gel electrophoresis or other separation technologies. In some embodiments, GC-MS is used to measure precursor enrichment level, and mass spectrometry is used to analyze the protein pool.

Suitable samples include all biological samples useful for determination of biomolecule turnover rates in subjects, including, but not limited to, cells, tissues (for example, lung, liver and kidney), bone marrow aspirates, bodily fluids (for example, blood, serum, urine, cerebrospinal fluid, bronchoalveolar levage, tracheal aspirates, sputum, nasopharyngeal aspirates, oropharyngeal aspirates, saliva), eye swabs, cervical swabs, vaginal swabs. Particularly suitable samples include blood samples, plasma samples, urine samples, serum samples, platelet samples, ascites samples, saliva samples and/or other body fluid samples, cells, a portion of a tissue, an organ, an isolated subcellular fraction, whole body, cellular sub-fractionations, muscle mitochondria, biopsy, or skin cell samples and the like.

In some embodiments, the disclosed methods include quantification to determine the fractional abundance of the one or more isotopomers of the at least one labeled biomolccule, for example quantification of the mass spec peaks at the half maximum.

In some embodiments, the disclosed methods include the application of heuristics to determine the quantifiability of the raw data. The interdiction of heuristics has the objective of determining quantifiability of mass isotopomers that have been identified. By applying several constraints to the data obtained this suitability can be determined. The first constraint is to fit the mass isotopomer time series data to first order decay equation:

□₀(□)=□₀(0)+{□₀(∞)−□₀(0)}(1−□⁻ [symbol] □)

The goodness of fit R² is calculated and data for the isotopomer is excluded if the fit is not above a certain threshold, which can be defined to the user. In some examples the R² threshold is great than 0.5%, such as greater than 0.5, 0.6, 0.7, 0.8. 0.9, 0.95 or even greater than 0.99, for example between 0.5 and 0.7, 0.8 and 0.9, 0.75 and 0.99. 0.8 and 0.95. The data can also be subject to the absolute constraint where 0<A₀(0)<1, and 0<A₀(∞)<1. The tolerance constraint is |A₀(t_min)−A₀(t_max)|>ϵ.

Where:

A₀(0) is predicted initial relative abundance.
A₀(∞) is predicted steady state relative abundance.
A₀(t_min) is predicted relative abundance at the earliest measured time point.
A₀(t_max) is predicted relative abundance at the latest measured time point.
ϵ is tolerance of mass spectrometer.

If a mass isotopomer time series data meets all three elements in the above criteria, that particular time-series data is considered to be quantifiable. In some embodiment, data that does not meet all three criteria is excluded from analysis.

In some embodiments, the criteria may further comprise a requirement based on the absolute area of the signal, or a requirement of the available number of data points, or adjustment of analysis parameters based on the variability of turnover rates between multiple fragments of the biomolecule.

In some embodiments, determining the biomolecule turnover rates of the one or more labeled biomolecules based on the fractional abundance of the one or more isotopomers comprises turnover rate determination based on kinetics of individual mass isotopomers. In some embodiments, the kinetic model comprises a first-order kinetic model of the precursor enrichment in the biological sample to predict the precursor enrichment level in a time-variable enrichment.

In some embodiments, determining the biomolecule turnover rates of the one or more labeled biomolecules based on the fractional abundance of the one or more isotopomers comprises a unified kinetic model that predicts biomolecule labeling behavior under both constant and time-variable precursor stable isotope enrichment.

In some embodiments, determining the biomolecule turnover rates of the one or more labeled biomolecules based on the fractional abundance of the one or more isotopomers further comprises a governing equation of both precursor enrichment rate and protein enrichment rate, and the use of nonlinear fitting optimization methods to directly calculate turnover rate from mass spectra.

In some embodiments, determining the biomolecule turnover rates of the one or more labeled biomolecules based on the fractional abundance of the one or more isotopomers further comprises modeling the number of labeling sites in the biological samples, the natural fractional abundance of the one or more isotopomer, and its plateau fractional abundance during and after labeling.

In some embodiments, samples can be subjected to 1-dimensional gel electrophoresis or other separation technologies. In some embodiments, GC-MS is used to measure precursor enrichment level, and mass spectrometry is used to analyze the protein pool.

### C. Labeling

To achieve stable labeling, subjects are administered an effective amount of ²H₂O. By effective amount, it is meant an amount sufficient to measure protein turnover rate. An effective amount can be a single bolus or administration over time or even a combination thereof, for example one or more boluses followed by administration over time. For example, it has been determined that to achieve stable labeling, mice can be given two intraperitoneal (IP) injections of 99% ²H₂O in saline 4 hours apart. The mice are allowed ad libitum access to 8% ²H₂O in drinking water after the first injection throughout the labeling period. At different time points (for example 0 d, 0.5 d, 1 d, 2 d, 4 d, 7 d, 12 d, 17 d, 22 d, 27 d, 32 d, 37 d, 90 d), mice are euthanized, and the serum, liver and heart are harvested, and the mitochondrial proteins are isolated by ultracentrifugation. While a specific protocol has been described, it is contemplated that the protocol can be altered by one of ordinary skill in the art given the amount of guidance presented in the specification, such that effective labeling is achieved.

In another non-limiting example, labeling in drosophila with a ²H enrichment of 8% in body water is achieved by adding 12% ²H₂O to the fly medium (agar/molasses/corn meal/yeast). The ²H enrichment level is designed to achieve efficient protein labeling without observable toxicity to the flies. Newly enclosed adult flies are transferred, cultured in the ²H₂O— containing fly medium. Flies are transferred to fresh, labeled media every 5 days, and harvested at 7 different time points (e.g., 0 d, 0.5 d, 1 d, 2 d, 4 d, 7 d, and 14 d after the initiation of labeling). The mitochondrial proteome, as well as proteins of other subcellular compartments, were stringently fractionated according to previously published protocols. While a specific protocol has been described, it is contemplated that the protocol can be altered by one of ordinary skill in the art given the amount of guidance presented in the specification, such that effective labeling is achieved.

In another non-limiting example, healthy human subjects are labeled by oral intake of ²H₂O, for example 60 mL of 70% ²H₂O three times per day for the first 7 days as the initiation period of labeling, followed by 50 mL of 70% ²H₂O twice a day for the next 7 days as the maintenance period of labeling. The maintenance period can be prolonged according to specific experimental purpose. Blood, urine, and saliva are collected to determine the ²H enrichment level in body water and the turnover rate of proteins. While a specific protocol has been described, it is contemplated that the protocol can be altered by one of ordinary skill in the art given the amount of guidance presented in the specification, such that effective labeling is achieved.

In another non-limiting example, heart failure patients are labeled by oral intake of ²H₂O, for example, 60 mL of 70% ²H₂O three times per day for the first 7 days as the initiation period of labeling, followed by 50 mL of 70% ²H₂O twice a day for the next 7 days as the maintenance period of labeling. The maintenance period can be extended according to specific experimental purposes. Blood, urine, saliva, and cardiac and adipose tissues when available, are procured to determine the ²H enrichment level in body water, the turnover rate of proteins, progression of disease, and response to treatments. A specific protocol has been described that has been approved by the UCLA IRB (Protocol #11-001053 and #12-000899), but it is contemplated that the protocol can be altered by one of ordinary skill in the art given the amount of guidance presented in the specification, such that effective labeling is achieved.

### D. Data Processing

Heavy water labeling has been demonstrated as an economical and viable alternative to other existing labeling methodologies. A particular advantage of heavy water is its ability to universally label all biosynthesized molecules. However, in part due to computational and software limitations, no work has explored the applicability of heavy water labeling for measuring the turnover rates of proteins, lipids, or metabolites in the omic scale. As disclosed herein the inventors have demonstrated that the development of the proper computational tools allows for a large-scale, high-throughput quantification of biomolecule turnover rates.

As disclosed herein, to facilitate the measurement of protein turnover rates on a proteomic scale, the inventors have developed a set of computational tools, named BioTurn, dealing with mass spectrometric data from ²H₂O labeling and have tested them in diverse biological systems. These computational tools fully automate all data processing steps, from the analysis of mass spectra to the determination of protein turnover rates. Finally, tracking the kinetics of the isotopic distribution provides a significant statistical advantage from the multiplicity of the mass isotopomers (m0, m1, m2, etc.).

A computational software package is developed to automate key steps in the analysis of raw mass spectrometric data. Advantages of the computational workflow include:

1. Automated quantification of MS peaks to determine the fractional abundance of mass isotopomers belonging to a peptide ion.

2. Heuristics introduced to determine quantifiability of MS raw data.

3. Turnover rates statistically inferred based on kinetics of individual mass isotopomers.

4. Multi-parameter fitting method allowing determination of turnover rate independent of steady-state enrichment level and circumvents the necessity for constant monitoring of ²H₂O enrichment level.

5. Data processed systematically with user-configurable parameters without the introduction of human bias.

6. Nonlinear model and computational optimization allowing determination of turnover rates from labeled protein samples taken from only one time point.

7. Nonlinear model and computational optimization allowing determination of turnover rates from labeled protein samples taken from different time points than the body water samples (taken for heavy water enrichment analysis).

8. Unified nonlinear model allowing determination of turnover rates from fast and slow enrichment experiments (e.g., mouse and human respectively) using identical methods.

9. Nonlinear model and computational optimization allowing a combined kinetic curve to be fitted to the experimental numerical values of peptide isotopomer fractional abundance.

In a labeling protocol where steady-state body water ²H enrichment is achieved (as described herein), the disclosed computational method is able to mathematically derive the parameters (both the initial and steady-state enrichment levels) required to calculate half-life. This circumvents the need to determine the level of ²H₂O molar percentage in body water using GC-MS, which has been a prerequisite in previous demonstration of mass isotopomer distribution analyses. The disclosed workflow is therefore streamlined, requiring less labor and instrumentation.

The demonstrated method, software, and labeling scheme enable for the first time the half-life of individual proteins to be determined in vivo in the scale of the whole tissue, cellular, or subcellular proteome, and that could be applied to multiple biological systems (multiple organs and organisms). Due to their limitations in data processing and throughput, previous ²H₂O labeling experiments were confined to either the measurement of total proteome turnover (irrespective of protein species) or the investigation of only few targeted proteins. To demonstrate the ability of this methodology, disclosed herein is use of heavy water labeling to investigate protein turnover in organelles such as the mitochondria, cytosol and nucleus; and in the cardiovascular system.

The high-throughput nature of a large-scale biomolecule turnover rate determination experiment necessitated the development of a computational platform to efficiently process raw mass spectra. In a non-limiting example, the ProTurn, a module within BioTurn designed for protein turnover analysis, was developed to address this need by providing a computer-implemented method to automate the process of peak detection, peak integration, mass isotopomer kinetics determination, and protein turnover kinetics determination. Analyzing heavy water enriched proteins includes determining the relative abundances of the individual mass isotopomers that compose the peptide ion. Retention time, mass, and charge state information from the peptide identification software is used to detect relevant features in the raw mass spectra. Peaks are detected using median-based thresholds, and the full-width at half-maximum of the extracted ion chromatogram's peak is integrated to determine the abundance. This process is repeated for subsequent mass isotopomers belonging to a given peptide ion, and the values are normalized across the mass isotopomers to compute the relative abundances.

Due to the inherent technical shortcomings of mass spectrometry, only a subset of mass isotopomers are quantifiable. To determine whether a mass isotopomer in an acquired spectrum is eligible for quantification and likely results from biological enrichment, ProTurn uses several heuristic rules. Firstly, given the experimental conditions of heavy water labeling, mass isotopomers over the course of time are expected to follow first order decay kinetics. A goodness-of-fit measure (R²) is used to determine how well a time-series data fits to this model, and only those mass isotopomers that have a R² greater than a defined threshold are retained for further quantification. Secondly, the measured changes in mass isotopomers relative abundance must be greater than the technical variability of the instrument. Thirdly, the extrapolated initial and steady-state information should be physically possible, i.e., the relative abundance of the isotopomer must be between 0 and 1. This serves as a fallback condition in the case that an unquantifiable mass isotopomer time-series data meets the two previous conditions. Because of the complex nature of mass spectrometry, these heuristics filter out mass isotopomer time-series data that are dominated by non-biological processes.

Heavy water-enriched proteins exhibit significant differences in the mass isotopomer distribution of the peptides from their natural state counterparts. Thus, the changes in the relative abundances over time of the individual mass isotopomers yield information on the turnover kinetics of the peptide as calculated from that particular mass isotopomer. However, in mass spectrometric analysis, individual proteins may contain multiple proteolytic peptides, and individual peptides contain multiple mass isotopomers. In order to consolidate these different types of data, relative abundances from a given mass isotopomer is transformed into fractional syntheses using extrapolated initial and steady state information. The resulting fractional synthesis data from all of the quantifiable mass isotopomers in a given protein is used to determine the turnover rate by a non-linear least-squares fitting to first-order decay kinetics. Alternatively, the median of the determined turnover rates of all the mass isotopomers in a given protein may be used to represent the protein turnover rate.

In a non-limiting example, the computer programs in the ProTurn module automatically determine the protein turnover rate from heavy water-enriched samples. ProTurn takes in as input raw mass spectra in mzML format, as well as protein identification information from search engines (e.g., SEQUEST and ProLuCID) and validation software (e.g., Scaffold). ProTurn will then generate an output, such as an Excel sheet containing the proteins and mass isotopomer data along with their corresponding turnover rates and other relevant quantities (e.g., errors and R²).

Disclosed herein is a computer-implemented method for determining the turnover rate of a biomolecule in subject, for example using one or more computing devices. Mass spectra data is received from samples collected from a subject at one or more time points, wherein biomolecules in the subject have been labeled with ²H. Biomolecule identification data is received and the mass spectra data and biomolecular identification data is parsed. The mass spectral data is assigned to the biomolecular identification data to identify peaks in the mass spectral data. The peaks in the mass spectral data is integrated to determine fractional abundance of one or more isotopomers of ²H labeled biomolecules in the samples. Enrichment rate and level data is received. The fractional abundance of the one or more isotopomers of ²H labeled biomolecules in the samples is fit to an equation describing labeled biomolecule turn over to determine the molecular turnover rates of biomolecules in the subject. In some embodiments, output of the molecular turnover rates of biomolecules in the subject is provided. In some embodiments of the method the mass spectral data is filtered to determine the quantifiability of the mass spectral data. Data that does not meet the criteria of quantifiability is removed from the analysis.

In some embodiments of the method, determining the biomolecule turnover rates of the one or more labeled biomolecules based on the fractional abundance of the one or more isotopomers comprises a unified kinetic model that predicts biomolecule labeling behavior under both constant and time-variable precursor stable isotope enrichment. In some embodiments of the method, the kinetic model comprises a first-order kinetic model of the precursor enrichment in the biological sample to predict the precursor enrichment level in a time-variable enrichment. In some embodiments of the method, determining the biomolecule turnover rates of the one or more labeled biomolecules based on the fractional abundance of the one or more isotopomers further comprises a governing equation of both precursor enrichment rate and protein enrichment rate, and the use of nonlinear fitting optimization methods to directly calculate turnover rate from mass spectra. In some embodiments of the method, determining the biomolecule turnover rates of the one or more labeled biomolecules based on the fractional abundance of the one or more isotopomers further comprises modeling the number of labeling sites in the biological samples, the natural fractional abundance of the one or more isotopomers, and its plateau fractional abundance during and after labeling. In some embodiments of the method, the biomolecule is a protein, nucleic acid, lipid, glycan, carbohydrate, or small molecule metabolite. In some embodiments of the method, the sample is a blood sample, a plasma sample, a urine sample, a serum sample, a platelet sample, an ascites sample, a saliva sample and/or other body fluid samples, a cell, a portion of a tissue, an organ, an isolated subcellular fraction, whole body, cellular sub-fractionations, muscle mitochondria, biopsy, or skin cell sample. In some embodiments of the method the subject is an organelle, a cell, or an organism.

A system and computer-executable program product that encompasses this method is also contemplated.

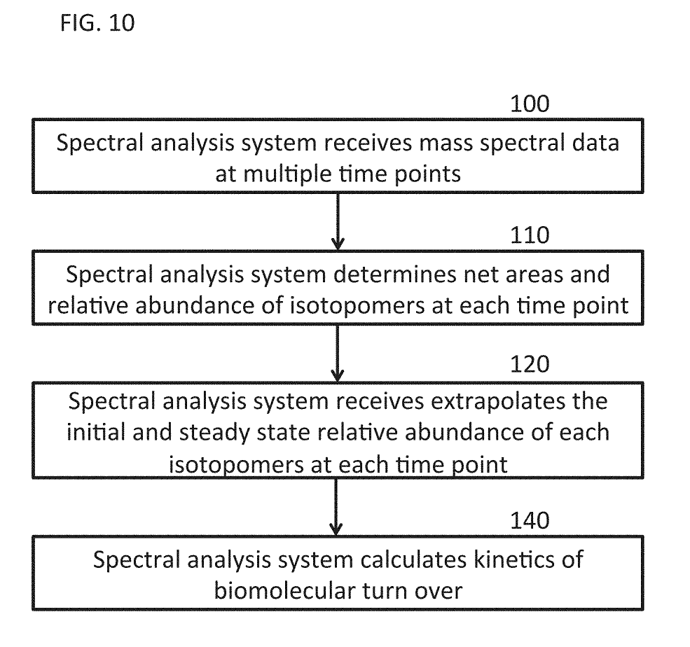

Aspects of the disclosed methods are described with reference to the flow described in the accompanying figures. With reference to FIG. 10, disclosed is a system for determining the turnover rates of biomolecules is a subject. In block 100 the spectral analysis system receives mass spectral data at one or more time points, such as multiple time points. In block 110, the spectral analysis system determines net areas and relative or fractional abundance of isotopomers at each time point. Using the net areas and fractional abundances of the determined isotopomers, at block 120, the spectral analysis system receives and extrapolates the initial and steady state relative abundance of each isotopomer at each time point. In some embodiments, in block 130 the spectral analysis system calculates kinetics of biomolecular turnover, for example using the methods described herein.

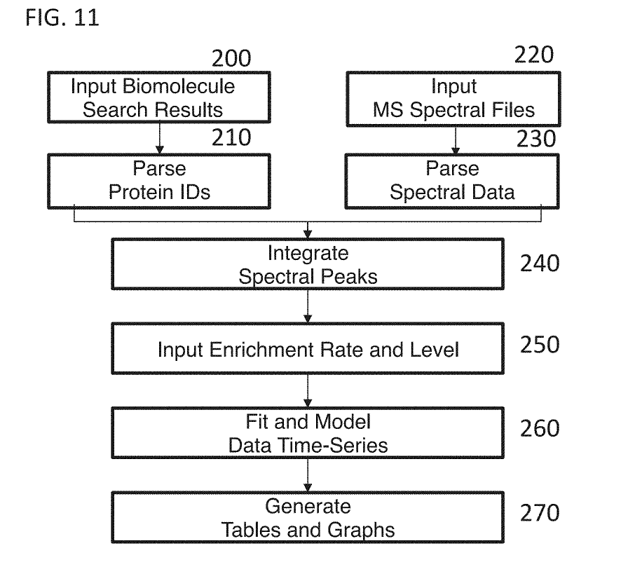

With reference to FIG. 11, disclosed is a system for determining biomolecule turnover rates in a subject. In block 200, the system receives input of biomolecule search results, such as protein search results for an organism of interest, for example proteins of interest. In block 210, the system parses the input of biomolecule search results to biomolecule IDs, such as protein IDs. In block 220, the system receives input of mass spectral files of a sample of interest, or multiple samples of interest, such as samples collected at one or more time points from a subject labeled with ²H. In block 230, the system parses the spectral data. In block 240, the system integrates the parsed (assigned) spectral peaks. In block 250, the system receives input of enrichment rate and level data for the assigned peaks. In block 260, the system fits the data, including the integrated peak data and the enrichment and level data to a model data time series to determine the biomolecule turnover rate of the assigned protein ids. In block 270, the system optionally generates table and graph of the biomolecular turnover rates, for example for inspection of the user. The results can then be compared.

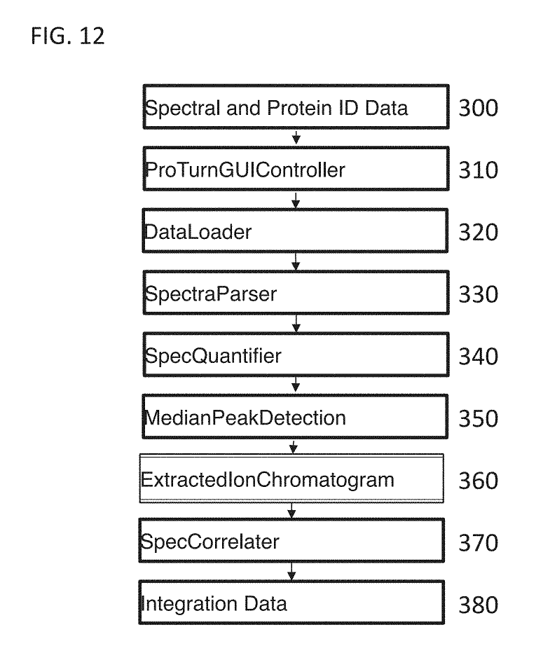

FIGS. 12-15 describe other aspects of the disclosed methods and systems. In block 300, a user locates the raw mass spectral and Protein ID data. In block 310, a user can select various parameters regarding the location and format of the file, for example using a graphical user interface, such as one controlled by the ProTurnGUIController module, in which to process the data. In block 320, the system reads the specific format of Protein ID as output by a typical search engine, such as using the DtaLoader, which reads the tab delimited text files from DTAselect to acquire information on the retention time and mass of each peptide, and saves the list of peptides and information to an input file. In block 330, the SpectralParser module parses the received spectral data, such as those stored in the [mzML] files, for example into individual spectra containing ions of particular mass, or individual peaks. In block 340, the SpecQuantifier module uses heuristic filters to determine if the parsed mass spectrometry data can be quantified and calls the block 350 MedianPeakDetection. In block 350, the MedianPeakDetection module generates a list of m/z values for the entire peptide envelop, using the identified peptide retention time and m/z information, then search the given collection of spectra to find all the present mass isotopomers of each identified peptide. This information is then used to call block 360, ExtractedIonChromatogram, to calculate the relative abundance of the isotopomers of the biomolecules of interest. In block 360, the ExtractedIonChromatogram module extracts the ions of interest, based on m/z information, over a time window in the mass spectrum chromatogram to create individual peaks of interest, and integrates them for areas. This data is stored in an areas array, with each index of the area representing data for each mass isotopomer. In block 370, the SpecCorrelater module gathers and links together the integrated peak area information for each corresponding peptide in every time point in the overall labeling experiment, which hitherto had been integrated separately. For example, for a particular peptide ion for example for the protein Q14624, this module finds the same peptide ion in day 0, day 1, day 2, day 3, and generates an array that stores the integration data together. In block 380, a user has at this point acquired fractional abundance time series of the mass isotopomers of interest for data fitting.

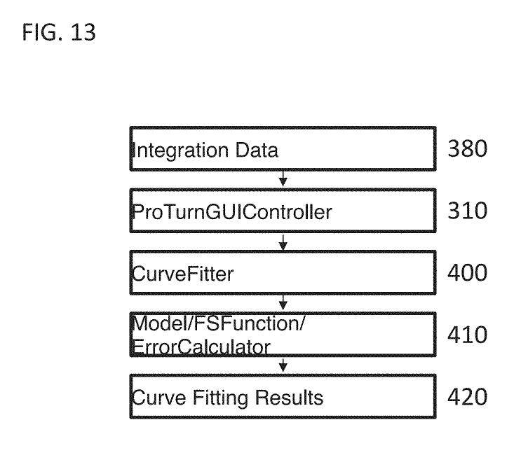

Turing to FIG. 13, in block 310, optional ProTurnGUIController module of the system receives the Integration Data 380. With the ProTurnGUIController module a user has the option of choosing parameters for curve fitting, for example whether to apply box-car or Savitzky-Golay smoothing, and the minimal time points the biomolecule must be identified in for it to qualify for fitting. These parameters are in turn used by CurveFitter module in block 400 to fit the integration data in block 380 to a kinetics model of choice, which may be a first-order exponential decay function (steady-state model), or a nonlinear, sigmoidal model (non-steady-state (NS) function). In block 400, the CurveFitter function performs multivariate optimization, such as using the Nelder-Mead method, and calls the Model/NSFunction modules in block 410. In block 410, the system applies the proper equation (based on user choice) to the nonlinear optimization process to minimize the error between the actual data point and the model function of choice and returns the best-fitted value of the parameter of interest (turnover rate). Block 410 also contains the ErrorCalculator module, which computes the error of estimate of the fitting process such as using nonlinear fitting (σA×dk/dA) or Monte Carlo method. The Curve Fitting Results can be output in block 420.

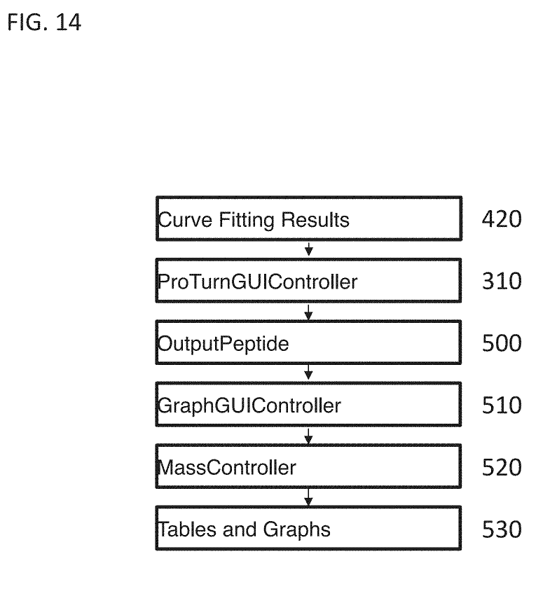

Turning to FIG. 14, Curve Fitting Results 420 are passed to optional ProTurnGUIController module 310. In block 500, OutputPeptide module tabulates the fitted turnover rate results (from each peptide isotopomer or each protein), which creates an interactive, sortable table controlled by block 510, optional GraphGUIController module. This allows a user to select an individual peptide, plot a mass isotopomer graph, such as through block 520, MassController module, and output the data through tables and graphs. In block 530, optionally tables and graphs are output, for example for inspection by a user.

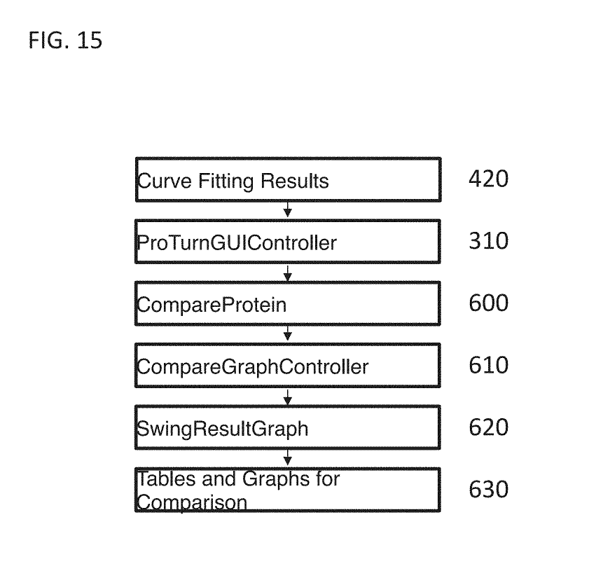

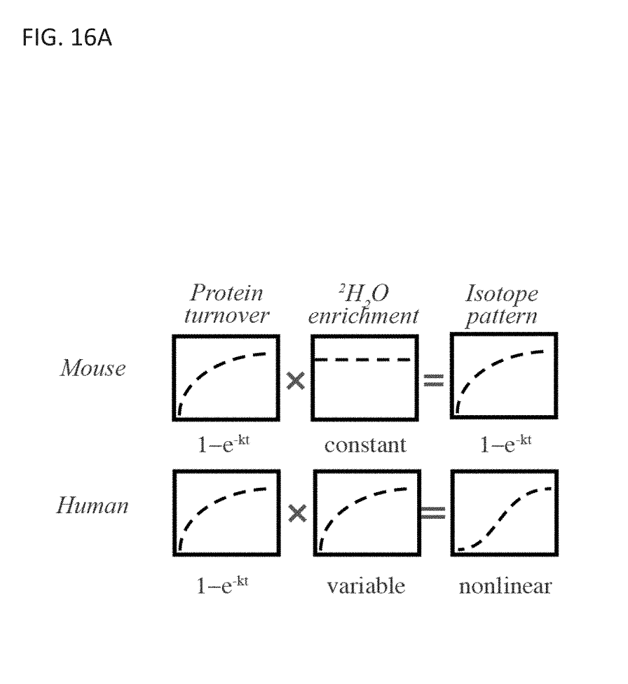

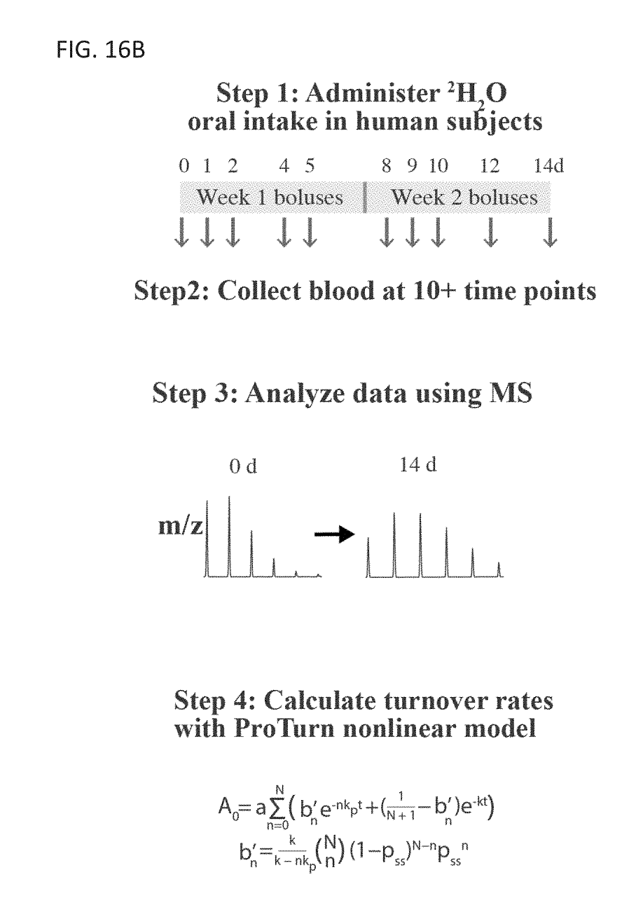

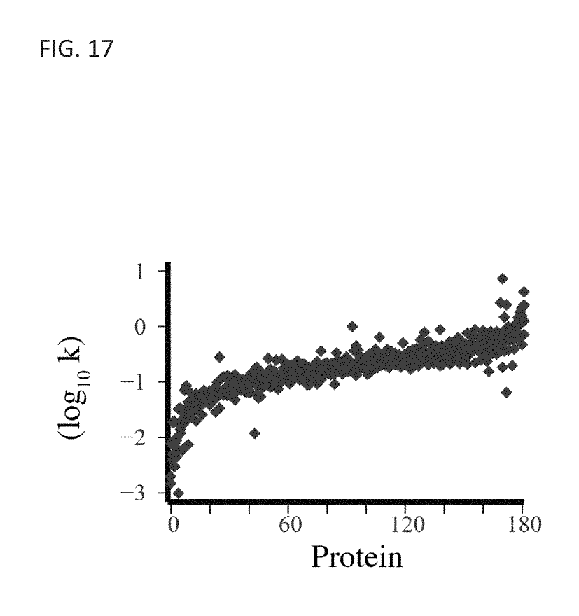

Turning to FIG. 15, Curve Fitting Results in block 420 are optionally passed to ProTurnGUIController module 310 to compare the turnover rates of more than one set of analyzed data. This function is handled by block 600, the CompareProtein module, to draw compare graphs and define table column properties (such as turnover rate ratio and statistical significance between two results). Block 610, optional CompareGraphController module, and block 620, SwingResultGraph module, together perform graphical drawing to provide further combined graphing capability, such as a kinetic curve showing the isotopomer fractional abundance from two different samples together. In block 630, optionally Tables and Graphs for Comparison is output, e.g., as a result to be inspected by a user.

### E. Metabolic Labeling in Time-Dependent Precursor Enrichment Model

The simpler, original equation for the relative abundance of M₀ isotopomer that we used for metabolic labeling in mouse is:

□₀(□)=□₀(0)+{□₀(∞)−□₀(0)}(1−□⁻ [symbol] ^{□})

Here, the relative isotopomer abundance at any given time, t, equals the sum of relative isotopomer abundance at time 0 and changes that come during the duration of labeling time.

A₀(∞) is the relative abundance when the peptide is fully labeled. Visually, this means the relative abundance reaches a plateau and undergoes no further change. Intuitively, A₀(∞) will be smaller than A₀(0) because we are looking at the monoisotopic peak, M₀. That is, the amount of this isotopomer relative to other isotopomer peaks will be less as time progresses.

The term {□₀(∞)−□₀(0)} is the full range of the relative abundance change during heavy water labeling. At any time point, the change toward the plateau value will follow first order kinetics. This is represented with the exponential terms.

The term, □₀(0), comes when we apply the boundary condition where at time 0, the relative abundance must equal to the natural abundance.

The exponential term comes from the integration of the first-order differential equation:

$$\frac{{dA}_{0}}{dt} = k [ {A}_{0} ( ∞ ) - {A}_{0} ]$$

The contribution of time-dependent precursor enrichment is represented by a new equation:

$${A}_{0} ( t ) = {A}_{0} ( 0 ) \cdot [ {e}^{- kt} + {\sum }_{n = 0}^{N} \frac{1}{1 - \frac{{k}_{p}}{k} ( N - n )} \cdot \frac{N !}{n ! ( N - n ) !} {( 1 - {P}_{ss} )}^{n} \cdot {P}_{ss}^{N - n} ( {e}^{- ( N - n ) {k}_{p} t} - {e}^{- kt} ) ] \qquad ( 0 )$$

To understand this equation, it is important to remember the following points about the precursor enrichment and turnover kinetics:

The precursor enrichment follows first order kinetics:

□=□ [symbol] _{□}(1−exp(−□ . . . □)) (1)

The precursor enrichment amount (Pₛₛ) is percentage of deuterium in body water at steady state (□=∞). This value is measured from the amount of heavy water we administrate to subjects after enough time has passed for the P to reach the plateau.

The term A₀(0) in the equation (0) is the relative isotopomer abundance of monoisotopic peak, M₀, at time 0; it is the natural relative abundance of any given peptide. This can be calculated based on the molecular formula of the peptide.

Ultimately, the relative abundance will reach the steady state value. This is represented as:

□₀(∞)=[□₀(0)](1−□) [symbol] (2)

Here we consider relative abundance of only the monoisotopic peak, M₀. So when we do not introduce precursor artificially, the maximum relative abundance of M₀ is simply □₀(0), the natural relative abundance. When we introduce isotopic precursor artificially at enrichment percentage (P) in the body water, the maximum relative abundance is correspondingly reduced according to the number of labeling sites and the percentage (P). (Here the percentage can be thought of as the probability of labeling at each site for all of the N sites.)

When we consider the dynamic change in the precursor enrichment percentage, we substitute equation (1) into (2). This yields:

□₀(∞)=[□₀(0)](1−□) [symbol] =[□₀(0)](1−□ [symbol] (1−exp(−□·□))) [symbol] (3)

One fundamental concept that we use to deduce the final equation (0) comes from the understanding that the change in the relative abundance follows first-order kinetics. That is,

$$\frac{{dA}_{0}}{dt} = {k}_{syn} - {k}_{deg} {A}_{0}$$

Given enough time for labeling, the relative abundance reaches a steady state or a plateau. The rate of change in

$${•}_{0} ( • \cdot • \cdot \frac{{••}_{0}}{••} )$$

is zero, so □ [symbol] is equal to □ [symbol] . In addition, since the relative abundance has reached a plateau, □₀ becomes a constant, which is better represented as the □₀(∞). Therefore, □ [symbol] =□ [symbol] □₀(∞).

The equation is then further simplified:

$$\frac{{••}_{0}}{••} = {•}_{•••} - {•}_{•••} {•}_{0} = {•}_{•••} [ {•}_{0} ( ∞ ) - {•}_{0} ] \qquad ( 4 )$$

When we replace the □₀(∞) from equation (3), the differential equation (4) becomes:

$$\frac{{••}_{0}}{••} = • ( [ {•}_{0} ( 0 ) ] {( 1 - {•}_{••} ( 1 - exp ( - {•}_{•} • ) ) )}^{•} - {•}_{0} ) \qquad ( 5 )$$

To integrate this equation, it is mathematically convenient to make a binomial expansion of the polynomial with the power of N. The binomial expansion reduces the power from N to a series of lower powers and involves sum of the combinations of terms within the polynomial:

$$\frac{{••}_{0}}{••} = • ( [ {•}_{0} ( 0 ) ] {\sum }_{• = 0}^{•} ( ( \begin{matrix}• \\ •\end{matrix} ) {( 1 - {•}_{••} )}^{• - •} {•}_{••}^{•} exp ( - {••}_{•} • ) ) - {•}_{0} ) \qquad ( 6 )$$

This equation can now be solved analytically by hand. The solution, presented as the final KL equation is:

$${A}_{0} ( t ) = {A}_{0} ( 0 ) \cdot [ {e}^{- kt} + {\sum }_{n = 0}^{N} \frac{1}{1 - \frac{{k}_{p}}{k} ( N - n )} \cdot \frac{N !}{n ! ( N - n ) !} {( 1 - {P}_{ss} )}^{n} \cdot {P}_{ss}^{N - n} ( {e}^{- ( N - n ) {k}_{p} t} - {e}^{- kt} ) ] \qquad ( 0 )$$

The relative abundance of the monoisotopic peak, M₀, at time t under time-dependent precursor enrichment is:

$${A}_{0} ( t ) = {A}_{0} ( 0 ) \cdot [ {e}^{- kt} + {\sum }_{n = 0}^{N} \frac{1}{1 - \frac{{k}_{p}}{k} ( N - n )} \cdot \frac{N !}{n ! ( N - n ) !} {( 1 - {P}_{ss} )}^{n} \cdot {P}_{ss}^{N - n} ( {e}^{- ( N - n ) {k}_{p} t} - {e}^{- kt} ) ] \qquad ( 0 )$$

Here, □₀(0) is the natural abundance of the monoisotopic peak. The e^{-kt} term comes from integration of the first order kinetics of protein turnover. The factorials, Pₛₛ^{N-n}, and (1−Pₛₛ)ⁿ terms, as well as the summation, come from the change in the precursor enrichment as time progresses. The denominator

$$\frac{1}{1 - \frac{{k}_{p}}{k} ( N - n )}$$

comes from the integration of the differential equation (6) that takes into account the first order kinetics of the protein turnover. The (N−n) term in the exponential, e^{-(N-n)k}^{ₚ}^{j}, comes from the binomial expansion when the precursor enrichment term raised to the N^{th} power was reduced to the sum of lower-powered terms. All these terms above together represent the contribution of the precursor enrichment rate to the final relative abundance of the monoisotopic peak.

Using this equation, each of the five parameters (kₚ, Pₛₛ, A₀(0), N, k) can be optimized by curve fitting the experimental data into the extended KL equation (0). One great advantage of this analytical solution is that we can understand the intrinsic behavior of each parameter from the model. In other words, we can know absolutely how the model will behave under any circumstances. This allows us more flexibility to apply the model in a variety of biological systems and conditions.

Note that the equation now fully describes the time-dependent change in A₀ as the result of labeling, and is a function of five parameters:
i. k, the turnover rate of the protein to which the peptide belongs. This is the parameter of interest.
ii. pₛₛ the plateau level of enrichment of ²H₂O in the biological system. This parameter can be readily measured with gas chromatography-mass spectrometry (GC-MS) from body fluid samples taken at a sampling time point after the ²H₂O level has reached steady state.
iii. kₚ, the rate constant of the rise-to-plateau kinetics of body water ²H₂O enrichment. This parameter can be deduced from fitting GC-MS measurements of body fluid samples at regular time points following the initiation of labeling to Equation S4.
iv. a, which represents the unlabeled fractional abundance of the 0^{th} isotopomer of the particular peptide. a can be readily calculated from the peptide sequence based on the natural biological abundance of heavy isotopes of carbon, nitrogen, oxygen, and sulfur, based on the formula:

□=(1−0.011)^{□}^{_{□}}(1−0.00366)^{□}^{_{□}}(1−0.00238)^{□}^{_{□}}(1−0.0498) [symbol] (S8)
N_{C}, N_{N}, N_{O}, N_{S} denote the number of carbon, nitrogen, oxygen, and sulfur atoms in the peptide, respectively.
v. N, which represents the number of deuterium-accessible labeling sites on the peptide sequence. N can be calculated as the sum of the known average accessible deuterium/tritium labeling sites on individual amino acids (N_{a a} ) in mice, as reported by Commerford et al. in the literature.

| Amino acid | Nₐₐ | | |
|---|---|---|---|
| (A) Alanine | 4.00 | (C) Cysteine | 1.62 |
| (D) Aspartate | 1.89 | (E) Glutamate | 3.95 |
| (F) Phenylalanine | 0.32 | (G) Glycine | 2.06 |
| (H) Histidine | 2.88 | (I) Isoleucine | 1.00 |
| (K) Lysine | 0.54 | (L) Leucine | 0.60 |
| (M) Methionine | 1.12 | (N) Asparagine | 1.89 |
| (P) Proline | 2.59 | (Q) Glutamine | 3.95 |
| (R) Arginine | 3.43 | (S) Serine | 2.61 |
| (T) Threonine | 0.20 | (V) Valine | 0.56 |
| (W) Tryptophan | 0.08 | (Y) Tyrosine | 0.42 |

The values for pₛₛ, kₚ, for an experiment, together with the values of a and N for each individual peptide, are then substituted into Equation 7, which can then be fitted using the Nelder-Mead method or the optimal value of k that minimizes the residual values between the model and the experimental data points.

The minimized sum of residual squares (σ_{A}) also allows the goodness-of-fit (R²) and the error of the fitting to be estimated (σₖ). For goodness-of-fit:

$${•}^{2} = 1 - \frac{{σ}_{•}}{{( {\sum }_{•} ( {•}_{0 , • = •} - \overline{•} ) )}^{2}} \qquad ( S9 )$$

In this study a more conservative filter and requiring R²≥0.9 and the peptide and its belonging protein to be explicitly identified in half the time points. In the mouse samples at least, where precursor enrichment is fast and peptide isotopomer time series follow a simpler first-order exponential decay curve, a fitting quality filter of R²≥0.8 appears to also accurately model the data and provide turnover rates without increased variability of k among peptides belonging to the same proteins.

The error of the fitting can be estimated by:

$$\begin{matrix}\frac{••}{{••}_{0}} {σ}_{•} = {σ}_{•} & ( S10 ) \\ \frac{••}{{••}_{0}} = \frac{1}{• {\sum }_{• = 0}^{•} ( \frac{{••}_{•}}{• ( • - {••}_{•} )} \frac{•}{• - {••}_{•}} {•}_{•} ( {•}^{- ••} - {•}^{- {••}_{•} •} ) - • ( \frac{1}{• + 1} - \frac{•}{• - {••}_{•}} {•}_{•} ) {•}^{- ••} )} & ( S11 ) \\ {σ}_{•} = \frac{{σ}_{•}}{• {\sum }_{• = 0}^{•} ( \frac{{••}_{•}}{• ( • - {••}_{•} )} \frac{•}{• - {••}_{•}} {•}_{•} ( {•}^{- ••} - {•}^{- {••}_{•} •} ) - • ( \frac{1}{• + 1} - \frac{•}{• - {••}_{•}} {•}_{•} ) {•}^{- ••} )} & ( S12 )\end{matrix}$$

Since Equation S12 is a function of time, it was opted to estimate the error where A₀ is most sensitive to the change of k among the time points where experimental data exist. In the figures, the upper bound and the lower bound of k are given by k+σₖ and k²/(k+σₖ), respectively.

Finally, one advantage of the method described herein is that since all parameters necessary to deduce turnover kinetics from mass isotopomer data are encompassed within a single equation, the sensitivity of turnover rates to errors in each parameter can be calculated precisely:

$${•}_{0} = • {\sum }_{• = 0}^{•} ( {•}_{•}^{′} exp ( - {••}_{•} • ) + ( \frac{1}{• + 1} - {•}_{•}^{′} ) exp ( - •• ) )$$

Derivatives of □

[symbol]

$$\begin{matrix}{•}_{•}^{′} = \frac{•}{• - {••}_{•}} ( \begin{matrix}• \\ •\end{matrix} ) {( 1 - {•}_{••} )}^{• - •} {•}_{••}^{•} \\ = \frac{•}{• - {••}_{•}} {•}_{•}\end{matrix}$$

$$\begin{matrix}\frac{{••}_{•}^{′}}{••} = ( \frac{1}{• - {••}_{•}} - \frac{•}{{( • - {••}_{•} )}^{2}} ) {•}_{•} \\ = - \frac{{••}_{•}}{{( • - {••}_{•} )}^{2}} {•}_{•} \\ = - \frac{{••}_{•}}{• ( • - {••}_{•} )} {•}_{•}^{′}\end{matrix}$$

$$\frac{{••}_{•}^{′}}{{••}_{•}} = \frac{••}{{( • - {••}_{•} )}^{2}} {•}_{•} = \frac{•}{• - {••}_{•}} {b}_{n}^{′}$$

$$\begin{matrix}\frac{{••}_{•}^{′}}{{••}_{••}} = \frac{•}{• - {••}_{•}} ( \begin{matrix}• \\ •\end{matrix} ) ( {• ( 1 - {•}_{••} )}^{• - •} {•}_{••}^{• - 1} - ( • - • ) {( 1 - {•}_{••} )}^{• - • - 1} {•}_{••}^{•} ) \\ = \frac{• - {••}_{••}}{{•}_{••} ( 1 - {•}_{••} )} {•}_{•}^{′}\end{matrix}$$

Derivative with Respect to □

$$\frac{{••}_{•}}{••} = • {\sum }_{• = 0}^{•} ( \frac{{••}_{•}^{′}}{••} exp ( - {••}_{•} • ) - • ( \frac{1}{• + 1} - {•}_{•}^{′} ) exp ( - •• ) - \frac{{••}_{•}^{′}}{••} exp ( - •• ) )$$

$$\frac{{••}_{•}}{••} = • {\sum }_{• = 0}^{•} ( \frac{{••}_{•}}{• ( • - {••}_{•} )} {•}_{•}^{′} ( exp ( - •• ) - exp ( - {••}_{•} • ) ) - • ( \frac{1}{• + 1} - {•}_{•}^{′} ) exp ( - •• ) )$$

Derivative with Respect to □_{□}

$$\frac{{••}_{0}}{{••}_{•}} = • {\sum }_{• = 0}^{•} ( \frac{{••}_{•}^{′}}{{••}_{•}} exp ( - {••}_{•} • ) - {•••}_{•}^{′} exp ( - {••}_{•} • ) - \frac{{••}_{•}^{′}}{{••}_{•}} exp ( - •• ) )$$

$$\frac{{••}_{0}}{{••}_{•}} = • {\sum }_{• = 0}^{•} ( \frac{•}{• - {••}_{•}} {•}_{•}^{′} ( exp ( - {••}_{•} • ) - exp ( - •• ) ) - {•••}_{•}^{′} exp ( - {••}_{•} • ) )$$

Second Derivative with Respect to □ and □_{□}

$$\frac{{•}^{2} {•}_{0}}{{••}_{•} ••} = • {\sum }_{• = 0}^{•} ( - \frac{•}{{( • - {••}_{•} )}^{2}} {•}_{•}^{′} ( exp ( - {••}_{•} • ) - exp ( - •• ) ) + \frac{•}{• - {••}_{•}} \frac{{••}_{•}^{′}}{□} ( exp ( - {••}_{•} • ) - exp ( - •• ) ) + \frac{••}{• - {••}_{•}} {•}_{•}^{′} exp ( - •• ) - •• \frac{{••}_{•}^{′}}{••} exp ( - {••}_{•} • ) )$$

$$\frac{{•}^{2} {•}_{0}}{{••}_{•} ••} = • {\sum }_{• = 0}^{•} ( - \frac{•}{{( • - {••}_{•} )}^{2}} {•}_{•}^{′} ( exp ( - •• ) - exp ( - {••}_{•} • ) ) + \frac{{•}_{2} •}{{• ( • - {••}_{•} )}^{2}} {•}_{•}^{′} ( exp ( - •• ) - exp ( - {••}_{•} • ) ) + \frac{••}{• - {••}_{•}} {•}_{•}^{′} exp ( - •• ) + \frac{{•}^{2} {•}_{•} •}{• ( • - {••}_{•} )} {•}_{•}^{′} exp ( - {••}_{•} • ) )$$

$$\frac{{•}^{2} {•}_{0}}{{••}_{•} ••} = • {\sum }_{• = 0}^{•} ( \frac{•}{{( • - {••}_{•} )}^{2}} {•}_{•}^{′} ( exp ( - •• ) - exp ( - {••}_{•} • ) ) + \frac{{•}^{2} {•}_{•}}{{• ( • - {••}_{•} )}^{2}} {•}_{•}^{′} ( exp ( - •• ) - exp ( - {••}_{•} • ) ) + \frac{••}{• - {••}_{•}} {•}_{•}^{′} ( exp ( - •• ) + \frac{{••}_{•}}{•} exp ( - {••}_{•} • ) ) )$$

Derivative with Respect to □_{□□}

$$\frac{{••}_{0}}{{••}_{••}} = • {\sum }_{• = 0}^{•} ( \frac{{••}_{•}^{′}}{{••}_{••}} ( exp ( - {••}_{•} • ) - exp ( - •• ) ) )$$

$$\frac{{••}_{0}}{{••}_{••}} = • {\sum }_{• = 0}^{•} ( \frac{• - {••}_{••}}{{•}_{••} ( 1 - {•}_{••} )} {•}_{•}^{′} ( exp ( - {••}_{•} • ) - exp ( - •• ) ) )$$

### E. Other Example Embodiments

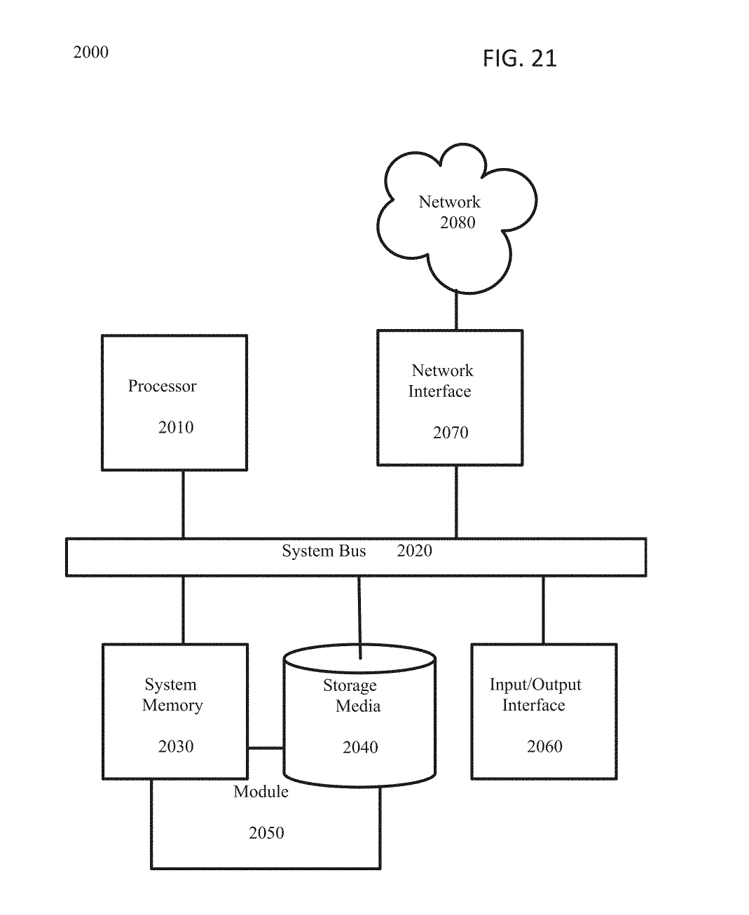

FIG. 21 depicts a computing machine 2000 and a module 2050 in accordance with certain example embodiments, for the determination of biomolecule turnover rates and/or half-lives of biomolecules, such as proteins in a cell or cells. The computing machine 2000 may correspond to any of any various computers, servers, mobile devices, embedded systems, or computing systems. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080. In some examples, the computing machine may be part of a mass spectrometer, connected to a mass spectrometer, and/or capable of receiving data from a mass spectrometer, such as through a network.

The computing machine 2000 may be implemented as a conventional computer system, an embedded controller, a laptop, a server, a mobile device, a smartphone, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor cure, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain example embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and/or associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the examples described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The following examples are provided to illustrate particular features of certain embodiments. However, the particular features described below should not be construed as limitations on the scope of the invention, but rather as examples from which equivalents will be recognized by those of ordinary skill in the art.

### EXAMPLES

### Example 1

This example describes methodologies used to determine the turnover rates of mitochondrial proteins in mice using the methods disclosed herein.

²H₂O Labeling of Mice and Tissue Collection

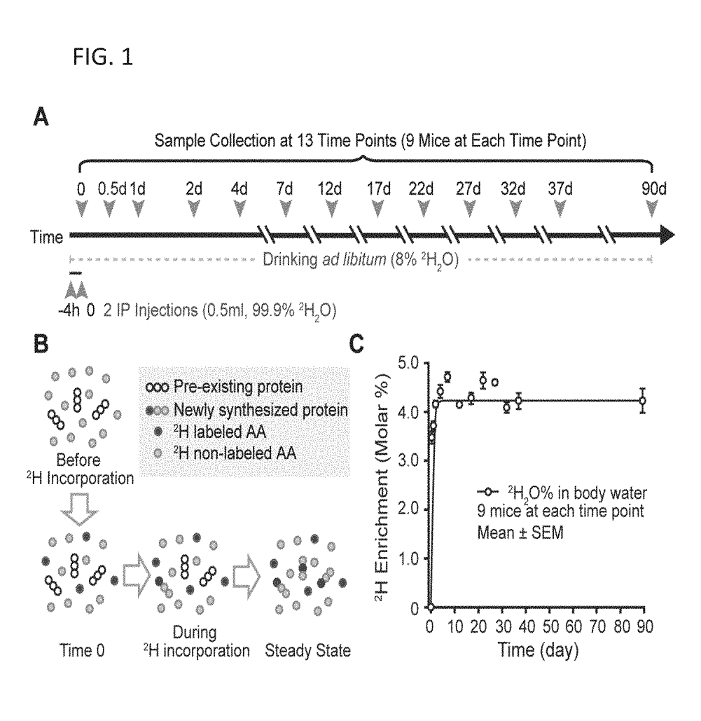

Male Hsd:ICR (CD-1) outbred mice (Harlan laboratories, 8-10 wk of age) were housed upon arrival in a 12:12 h light-dark cycle with controlled temperature and humidity, free access to standard lab chow and natural water. No significant change was observed in body weights of mice (˜40 g) during the labeling period. ²H₂O labeling was initiated by two IP injections of 99.9% saline ²H₂O (Cambridge Isotope Laboratories) spaced by 4 h, then mice were allowed free access to 8% ²H₂O to maintain a steady-state labeling level at ˜4.3% in body water (FIG. 1 (A)). Heart, liver, and blood were harvested at 13 time points (0, 0.5, 1, 2, 4, 7, 12, 17, 22, 27, 32, 37, and 90 d) from the second IP injection (t=0). At each time point, 3 groups of 3 mice each were euthanized. All 3 groups from each time point were used to determine the extent of ²H labeling in body water; one group was used to calculate protein turnover rates.

### GC-MS Analysis of Serum Water

²H labeling in body water was measured by GC-MS after exchange with acetone as described (McCabe, et al., Anal. Biochem. 350, 171-176, 2006). Serum was centrifuged for 20 min at 4,000 rpm at 4° C., and 20 μl of serum or ²H₂O standard for calibration curve was reacted with 2 μl of 10 N NaOH and 4 μl of 5% (v/v) acetone in acetonitrile. After overnight incubation at ambient temperature, acetone was extracted by adding 500 μl of chloroform and 0.5 g of anhydrous sodium sulfate, and 300 μl of the extracted solution was aliquoted and analyzed on a GC mass spectrometer (Agilent, 6890/5975) with a DB17-MS capillary column (Agilent J&W, 30 m×0.25 mm×0.25 μm). The column temperature gradient was as follows: 60° C. initial, 20° C./min increase to 100° C., 50° C./min increase to 220° C., 1 min hold. The mass spectrometer operated in the electron impact mode (70 eV) and selective ion monitoring at m/z 58 and 59, with 10 ms dwell time.

### Isolation of Cardiac and Hepatic Mitochondria

Mitochondria were isolated by ultracentrifugation as described (Zhang et al., Proteomics 8, 1564-1575, 2008). Hearts and livers were excised from euthanized mice, homogenized in the homogenization buffer (250 mmol/l sucrose, 10 mmol/l HEPES, 10 mmol/l Tris-HCl, 1 mmol/l EGTA, protease inhibitors (Roche Complete, 1×), phosphatase inhibitors (Sigma Phosphatase Inhibitor Cocktail II and III, 1×), and 10 mmol/l of dithiothreitol (Sigma), pH 7.4), then centrifuged at 800 rcf at 4° C. for 7 min. The supernatant was centrifuged at 4,000 rcf at 4° C. for 20 min. The pellets were washed, centrifuged again, then resuspended in 19% (v/v) Percoll (Sigma) in the homogenization buffer, overlaid on 30% and 60% Percoll, and ultracentrifuged at 12,000 rcf at 4° C. for 20 min to remove microsomes. Purified mitochondria were collected from the 30%/60% Percoll interface, washed twice, centrifuged at 4,000 rcf at 4° C. for 20 min, then lysed by sonication in 10 mmol/l Tris-HCl, pH 7.4.

Electrophoresis and in-Gel Digestion of Proteins

Mitochondrial proteins were separated by sodium dodecyl sulfate-polyacrylamide gel electrophoresis (SDS-PAGE); 200 μg of proteins were denatured at 70° C. in Laemmli sample buffer for 5 min, then separated on a 12% Tris-glycine acrylamide gel with 6% stacking gel, at 80 V, at ambient temperature for ˜19 h. The gel was Coomassie-stained and cut into 21 fractions. Each fraction was digested with 30:1 (w/w) sequencing-grade trypsin (Promega) following reduction and alkylation by dithiothreitol and iodoacetamide (Sigma), respectively.

### LC-MS and MS/MS

Peptide identification and mass isotopomer quantification were performed on an LTQ Orbitrap XL mass spectrometer (ThermoFisher Scientific), coupled to a nanoACQUITY UPLC system (Waters). The trapping (30 mm) and analytical (200 mm) columns for peptide separation were packed in IntegraFrit columns (New Objective, 360 μm O.D., 75 μm I.D.) using Jupiter Proteo C₁₂ resin (Phenomenex, 90-Å pore, 4-μm particle). The binary buffer system consisted of 0.1% formic acid in 2 and 80% ACN for buffer A and B, respectively. The separation gradient was made by changing buffer B: 0 min—2%, 0.1 min—5%, 70 min—40%, 90 min—98%, and 100 min—98%, and 105 min—2% with subsequent equilibration at 2% for 5 min. Mass spectra were obtained in profile mode for MS survey scan in the Orbitrap at a resolution of 7,500 and in centroid mode for MS/MS scan in the LTQ. The top 5 intense peaks in the MS scan were subjected to CID with an isolation window of 3 Thomson (Th) and dynamic exclusion of 25 seconds.

### Database Search for Protein Identification

The raw data were processed by BioWorks (ThermoFisher Scientific, version 3.3.1 SP1), and searched using SEQUEST (ThermoFisher Scientific, version 3.3.1) against the UniProt mouse database (Jul. 27, 2011; 55,744 entries). Search parameters included fixed cysteine carbamidomethylation and variable methionine oxidation, trypsin enzymatic specificity, and two missed cleavages. The mass tolerances for the precursor and the product ions were 100 ppm and 1 Th, respectively. The minimum redundancy set of proteins was acquired with Scaffold (Proteome Software, version 3.3.3). At least 2 peptides and 99.0% protein confidence were required for protein identification, and the global false discovery rate was 0.1%. Peptides shared by multiple proteins or protein isoforms were excluded from downstream turnover rate calculations.

### Quantification of Mass Isotopomers

²H in body water is metabolically incorporated into the C—H bonds of free non-essential amino acids by multiple enzymes. Unlike labile N—H or O—H bonds, the C—H bonds are stable and the incorporated ²H in non-essential amino acids do not back-exchange during sample processing. Additionally, H in the α-carbon of essential amino acids is reversibly accessible to ²H by transamination. The ²H-labeled amino acids are integrated into newly synthesized protein via t-RNAs, and with each cycle of turnover, into proteins until their ²H content reaches steady-state equilibrium with surrounding ²H₂O. The rate of protein turnover is determined by tracking the time evolution of mass isotopomer distributions (FIG. 1 (B)). To accommodate the determination of protein turnover rate on a proteomic scale, the inventors developed a software package, BioTurn. One of its modules, ProTurn, was designed to quantify the peptide ion mass isotopomer distribution, and subsequently perform curve-fitting to determine rate constants of protein turnover. RAW files were converted into mzML format by Proteo Wizard (version 2.2.2913) for input. ProTurn obtains the extracted ion chromatogram (XIC) for each identified peptide ion using retention time and a mass isolation window of ±100 ppm. Then, the peak area under the XIC is integrated to determine the normalized abundances of all mass isotopomers corresponding to a peptide ion. At any given time point (t), the normalized peak area for a designated mass isotopomer, [symbol] (□), is determined by dividing the peak area (□) of the mass isotopomer □, i.e., [symbol] (□), over the summation of peak areas from all mass isotopomers (Σ [symbol] ₌₀□_{□}(□)):

□_{□}(□)=□_{□}(□)/Σ [symbol] ₌₀□ [symbol] (□) (Eq. 1)

where Iⱼ is the peak area of the mass isotopomer mⱼ (f=0, 1, 2, . . . , N).

### Calculation of Protein Turnover Rates

To determine protein turnover rate, the normalized peak intensities at t=0, A(0), and at full enrichment, A(∞), were defined from the time-series data of each mass isotopomer by non-linear fitting into a first-order kinetics equation:

□(□)=□(0)+{□(∞)−□(0)}(1−□⁻ [symbol] ) (Eq. 2)

where k is the rate constant, which describes the rate at which proteins are newly synthesized to replace the existing pool. Assuming equilibrium, it equals the rate at which proteins are degraded. Subsequently, the time-series data of all mass isotopomers from a protein were transformed into fractional synthesis, f(t), which is the fraction of total protein newly synthesized through turnover, by rearranging Eq. 2;

□(□)={□(□)−□(0)}/{□(∞)−□(0)}=1−□⁻ [symbol] (Eq. 3)

ProTurn excluded data from the curve fitting with a R² value less than 0.7 or containing less than 5 time points from the calculation of fractional synthesis. The chosen R² value of 0.7 was adjudged empirically to balance high accuracy and precision in the measurement of the kinetic data. As A(0) and A(∞) are theoretically bound between 0 and 1, only experimental values between −0.1 and 1.1, in consideration of experimental errors, were included in fractional synthesis calculation. Finally, fractional syntheses from all of the mass isotopomers corresponding to a particular protein were fitted to the first-order kinetics equation (Eq. 3) to determine k for protein turnover.

### Statistical Analyses

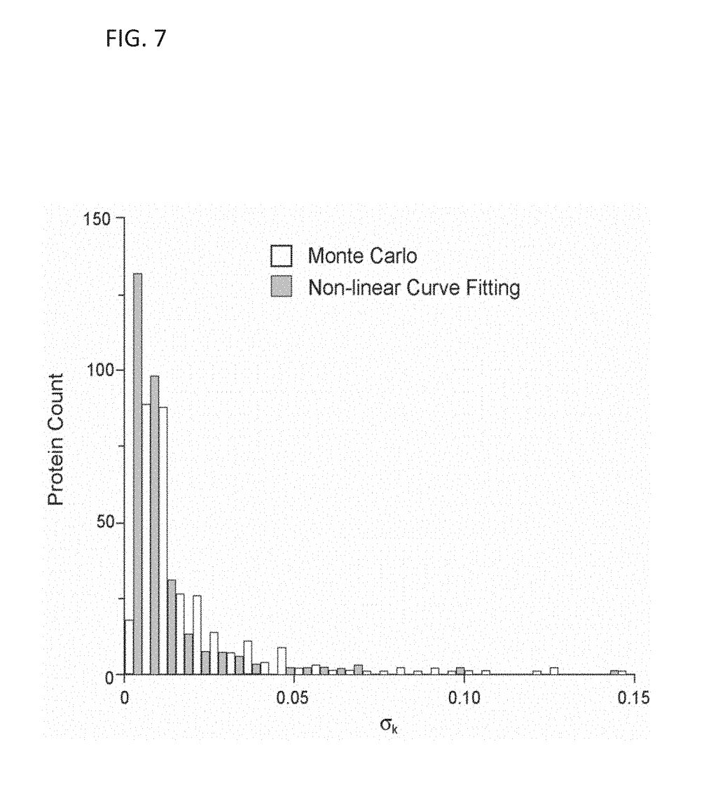

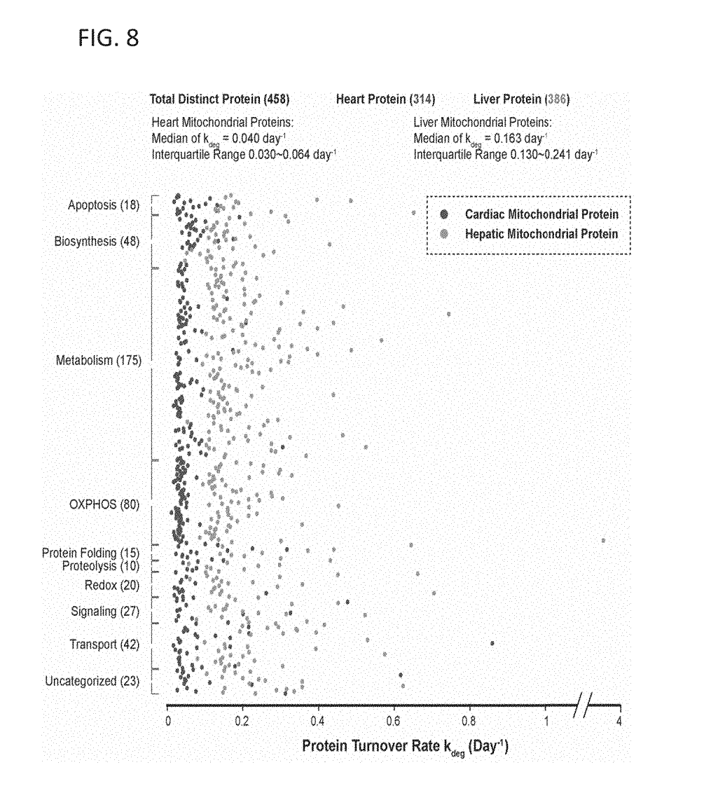

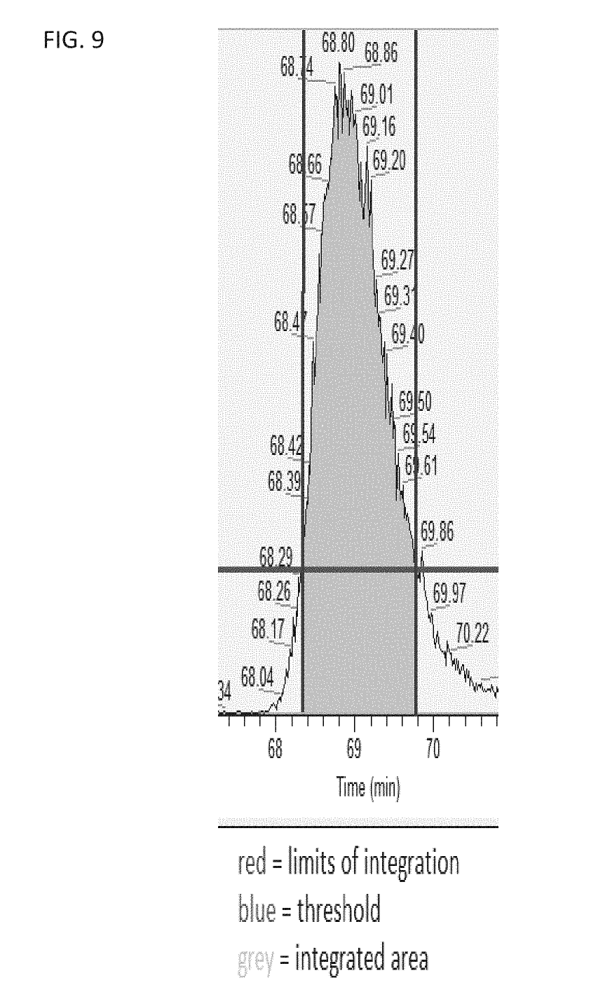

Uncertainties in rate constants were estimated using the Monte Carlo method. The distribution of the relative abundance was approximated using the absolute value of the residuals. At each measured time point, a single point was synthetically generated using random numbers from a Gaussian distribution with the same width as the distribution of the absolute values of the residuals and a mean of the model value. New rate constants were determined for the 10,000 synthetic datasets, and the distribution of rates was observed to converge approximately to a Gaussian distribution. The width of this distribution (1σ) was reported as the standard error of the rate constant (In principle, there is little difference in the standard error estimation between the Monte Carlo and Non-linear curve fitting methods. For comparison, the histograms of the errors in the rate constants for cardiac proteins are given in FIG. 7). Quantile-quantile plots clearly suggest that degradation rates of proteins within an organ were not normally distributed. Significances of difference between groups were thus assessed by the rank-based, non-parametric Mann-Whitney U test using R. Correlations between variables were denoted by Spearman's rank-correlation coefficient (ρ).

### Results

Precursor Enrichment in Serum During ²H₂O Labeling

Fractional protein synthesis is calculated based on the precursor-product relationship, which states that product labeling enrichment would reach that of the precursor at steady state. To quantify the level of precursor ²H incorporation during labeling, the serum of mice was sampled at all experimental time points. As water quickly equilibrates throughout the body and permeates cellular compartments, water in the serum serves as a proxy for ²H incorporation in all organs. GC-MS experiments measured the molar percentage of ²H in serum water, which rapidly reached 3.5% within 12 h following two IP injections of 99.9% ²H₂O (FIG. 1 (C)). Throughout the labeling period, ad libitum feeding of 8% ²H₂O maintained ²H enrichment at ˜4.3% (FIG. 1 (C)). The speed and stability of ²H incorporation in the experiment support the calculation of fractional synthesis from constant precursor enrichment.

### Time Evolution of Mass Isotopomer Abundance Distribution of ²H-Labeled Peptides

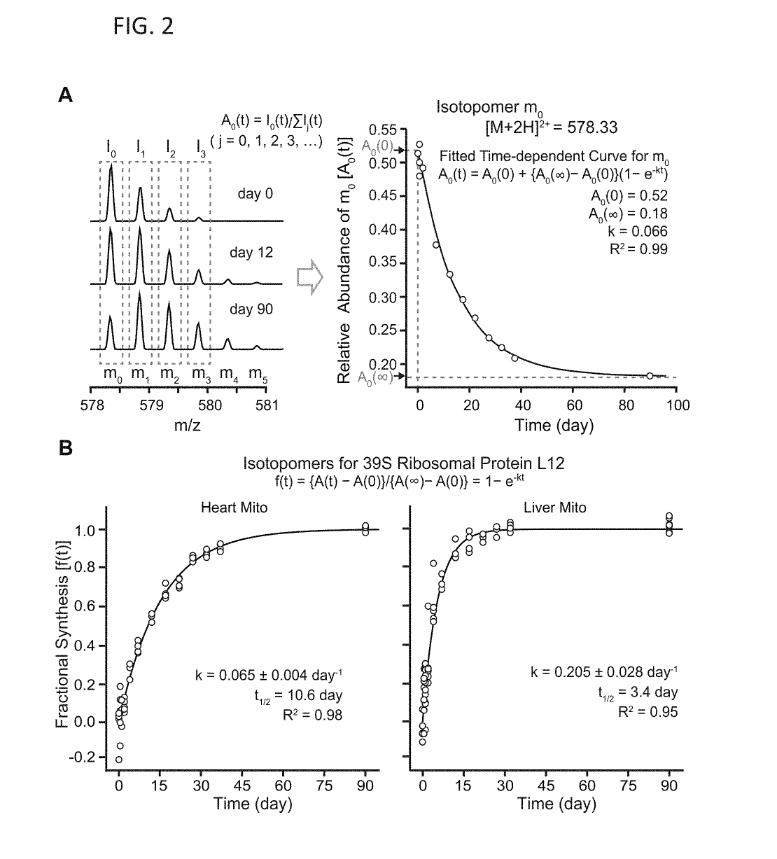

Mass isotopomer distributions of peptide ions change over time as ²H is introduced from the precursor pool into the protein pool through protein turnover. FIG. 2 (A) displays the temporal profile (0 to 90 d) of mass isotopomer distributions for a given tryptic peptide [M+2H]²⁺=578.33 m/z, from the mitochondrial 39S ribosomal protein L12 (MRPLI12). Prior to ²H₂O labeling (0 d), the first mass isotopomer (m₀) gave the most intense peak. When the labeling time reached 12 d, the peak intensity of m₀ became comparable to that of m₁, and one new feature corresponding to m₄ was observed. After 90 d of labeling, m₀ became the third most intense mass isotopomer and the high-mass isotopomer peak m₅ appeared. In summary, ²H₂O labeling resulted in anticipated changes in isotopomer peak intensity that allow protein fractional synthesis to be calculated. Accordingly, the inventors proceeded with the proteome scale characterization of protein turnover from the heart and livcr mitochondria isolated from the same animals.

The intensities of mass isotopomers were quantified by ProTurn, which integrated the areas-under-peak in the XIC, then normalized the peak area of each isotopomer by the summed intensity of all isotopomers in that particular peptide ion to determine its relative abundance (Eq. 1). For every mass isotopomer with quantification data at five or more time points, the relative abundances from all time points were fitted to an exponential decay equation (FIG. 2 (A)). For a particular mass isotopomer, multiple normalized peak intensities may exist due to the detection of identical peptides in multiple gel bands, different charge states, or oxidized forms. Identical isotopomers from multiple gel bands were combined, but those of different charge states or oxidized forms were fitted independently. The fitting is extrapolated to yield the normalized abundance of the mass isotopomer at its initial, A(0), and steady, A(∞), states.

In summary, two distinct criteria were applied for peptide selections. The first concerns with the protein identification, where Scaffold was used to validate and filter peptides based on their confidence levels. The second addresses the precision of curve fitting by using a R² threshold filter. Mass isotopomers that met these two criteria were accepted for protein turnover rate calculations.

### Rules of Protein Turnover in Cardiac and Hepatic Mitochondria

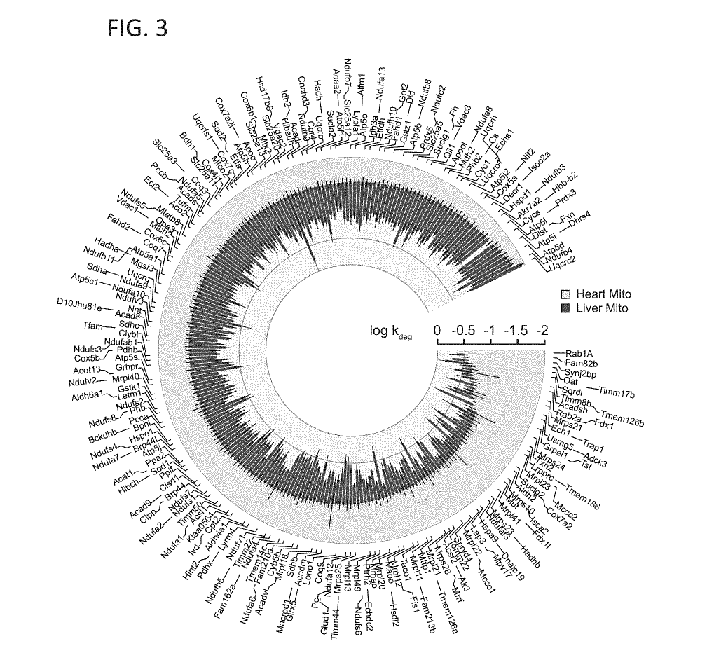

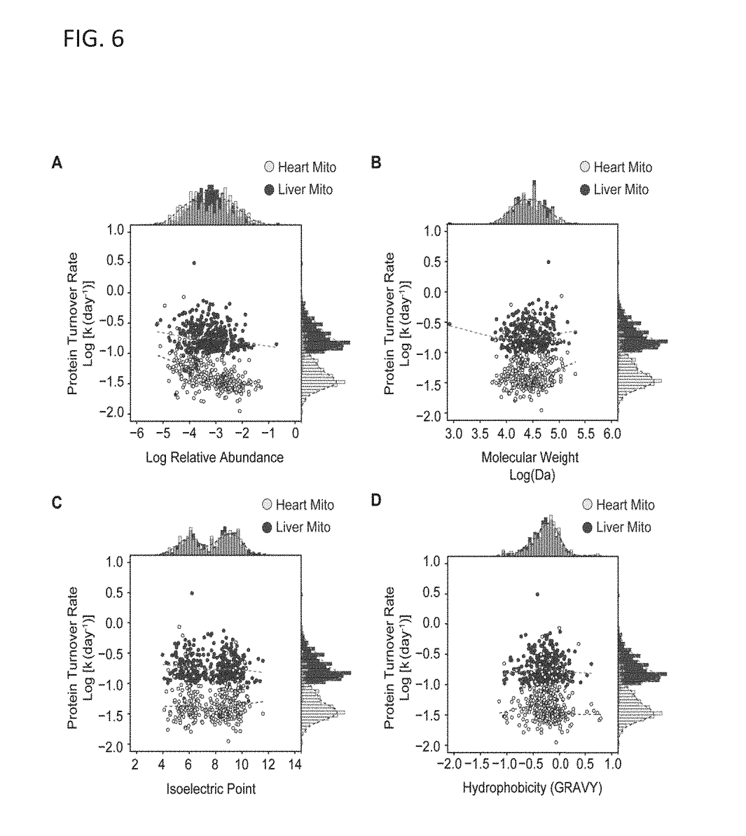

From the fitted A(0) and A(∞) values, all isotopomer data of a protein were transformed into protein fractional synthesis by Eq. 3. For m₀ at some early time points, experimentally measured A(t) could be larger than the computationally determined A(0) due to the fitting error, which leads to a negative fractional synthesis value. It was found that filtering by R² value at this point excluded unquantifiable isotopomers and improved the accuracy of turnover rate calculation without significantly impacting the number of analyzed proteins. FIG. 2 (B) shows an example of fractional synthesis time evolution from the mitochondrial 39S ribosomal protein L12. The fractional synthesis data were fitted to an exponential curve to yield the protein turnover rate, k. The 39S ribosomal protein L12 turns over at the rate of 0.065±0.004 d⁻¹ (R²=0.98) in the heart, but almost three times faster in the liver, at 0.205±0.028 d⁻¹ (R²=0.95) (FIG. 2(B)). Such differences in turnover rates were generally observed between mitochondrial proteins in the heart and in the liver; median turnover rate was about 4 times higher in the liver than in the heart (0.040 d⁻¹ and 0.16 d⁻¹). With the exception of 3 proteins (MRPS24, RAB1A, and SYNJ2BP), all 242 commonly analyzed proteins demonstrated slower turnover (i.e. longer half-life) in cardiac mitochondria (FIG. 3). In total, the turnover was deduced for 314 proteins in cardiac mitochondria and 386 in hepatic mitochondria, among which 458 are distinct. This study captured mitochondrial proteins in all major functional categories, spanning 5 orders of magnitude in protein abundance (see FIG. 6).

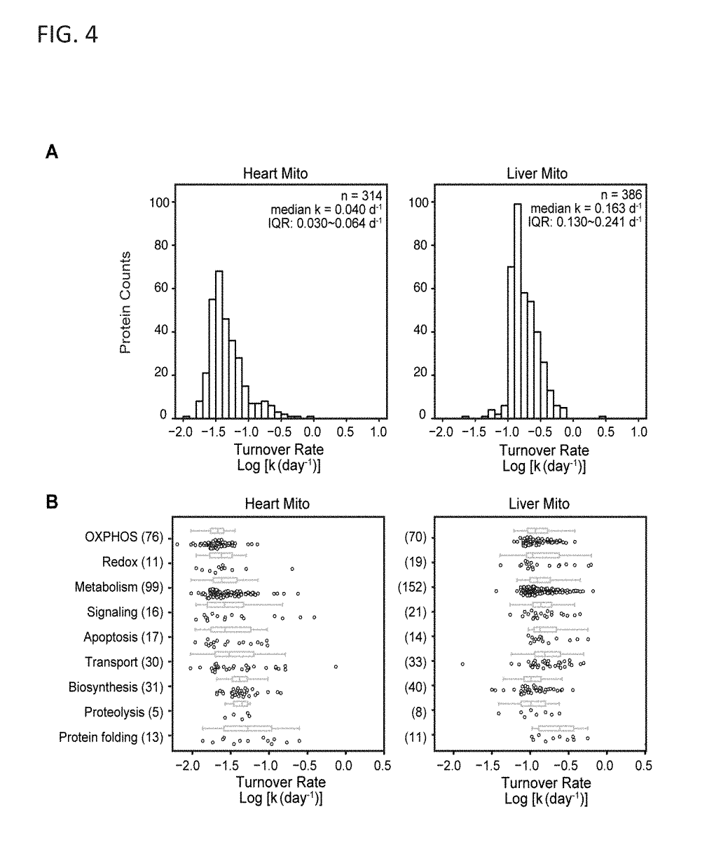

FIG. 4 (A) shows the distribution of turnover rates in the analyzed proteins in the liver and the heart. The analyzed protein kinetics range over 2.4 orders of magnitude in total, and spanned 1.8 and 2.2 orders of magnitude in the heart and the liver, respectively. Between the 5^{th} and 95^{th} percentiles, protein turnover rates differed by 7.9-fold in the heart and 4.3-fold in the liver. To determine whether the observed turnover rates correlate with biological functions, the observed cardiac and hepatic mitochondrial proteins were categorized by Gene Ontology (FIG. 4 (B)). In both tissues, proteins associated with protein folding showed relatively faster turnover, while those related to redox turned over rather slowly. By contrast, proteins involved with biosynthesis and proteolysis displayed disparate turnover between the two tissues. Biosynthesis proteins had fast turnover in the heart but not in the liver. However, significant overlaps in turnover rates were observed among the functional categories in both the liver and the heart.

### Discussion

As demonstrated herein a novel strategy has been developed utilizing ²H₂O labeling to examine the kinetics of mitochondrial proteins in mouse heart and liver on a large scale. The computational approach created automated the characterization of fractional protein synthesis and deduced protein half-life without steady-state isotopomer abundance information. With this integrated platform of MS and informatics, the inventors successfully obtained the turnover rates of 458 proteins in mouse cardiac and hepatic mitochondria.

### Data Analysis

In the disclosed method, the plateau ²H-enrichment in the peptide was computationally deduced from the experimental data points. In addition, all data processing was fully automated, enabling the limitations in throughput to be overcome.

In analyzing the large-scale set of data points, the inventors took the following considerations to address the experimental errors, which may be contributed by multiple sources. Firstly, the experimental error is directly linked to experimental conditions, including the reliability in peak area measurement, the separation of overlapped chromatographic peaks, spectral accuracy, and absolute peak intensities. Secondly, this study takes the assumption of first-order kinetics in its curve fitting to extract the kinetic information, under the scenario where this kinetics is forced, a larger error will result. Ostensibly, the first-order kinetics model used in this study does not hold homogeneously for all experimental data. In other words, proteins whose turnover deviated from first-order kinetics would be fitted with a larger error. Thirdly, the inventors filtered out redundant peptides from known protein isoforms to ensure that only unique peptides were selected for individual proteins, and to avoid ambiguity in the protein kinetics calculation. However, peptides shared by either undocumented or undiscovered isoforms may remain, subsequently causing an increased error formation in data processing.

Extensive fractionation and enrichment procedures were conducted to yield functionally viable mitochondria. The very majority of the detected proteins are classically established mitochondria proteins. However, some identified proteins may be classified as mitochondria-associated proteins, whereas some non-mitochondrial contaminants inevitably remain in a mitochondrial isolation. Because of the stringent criteria established by the inventors in filtering both protein identification and turnover data, common contaminant proteins (e.g., keratin) were automatically expunged from the final kinetic data. The inventors surmise that among the 458 analyzed proteins, there is 1 protein representing a highly likely non-mitochondrial contaminant (Hemoglobin subunit beta-2, HBB-B2). Incidentally, the approach developed by the inventors detected almost identical turnover rates (k=0.021 d⁻¹) for only HBB-B2 in liver and heart mitochondria, which suggests the shared blood origin of the HBB-B2 protein from the two independent experiments. These data independently validate the reproducibility of the technology platform disclosed herein.

### Rules Governing Turnover Rate of Proteins

Previous metabolic labeling studies suggested protein turnover rates differ across mammalian organs. The results demonstrate such tissue-specific differences are preserved in the mitochondrial protcome (FIG. 3), supporting the hypothesis that intra-genomic differences in organ phenotypes directly constrain mitochondrial protein dynamics. The turnover rates cannot be explained by liver cell turnover, as mouse liver DNA has half-life exceeding 300 d (Commerford et al., Proc. Natl. Acad Sci. U.S.A. 79, 1163-1165, 1982). Secondly, the median mitochondrial liver protein turnover rate agrees with the reported values of gross mitochondrial turnover rates (Miwa et al., Aging Cell, 7, 920-923, 2008).

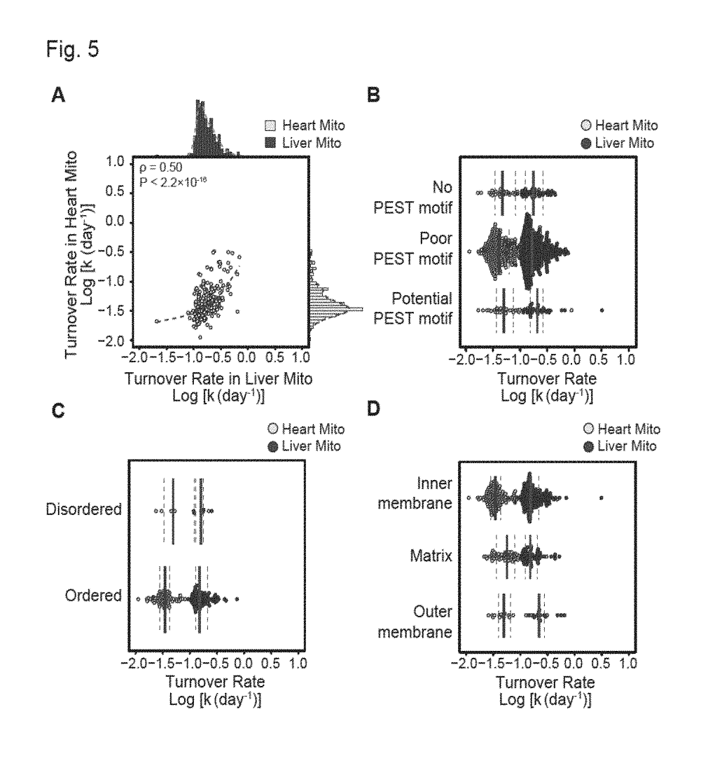

The inventors observed a good correlation between protein turnover rates in the heart and in the liver (Spearman's ρ=0.50, P<2.2×10⁻¹⁶) (FIG. 5 (A)), suggesting the determined distribution of protein turnover is robust. However, the correlations are not without exceptions, which indicates additional laycrs of regulatory mechanisms. Several models were proposed in the literature to explain the diversity in turnover rates, either within mitochondria or across the whole cell. The inventors further investigated whether some of these intrinsic protein properties may account for the turnover rates in their large-scale dataset. The presence of the PEST motif and intrinsic protein sequence disorder have been proposed as determinants of protein kinetics. It was found that there was no proteome-wide evidence of distinct turnover for both features in either organ (Mann-Whitney U test, P>0.05) (FIGS. 5 (B and C)). By contrast, the data here support previous observations that proteins on the outer mitochondrial membrane turn over faster than those on the inner membrane (Mann-Whitney U, Heart: P=5.55×10⁻³, Liver: P=5.21×10⁻⁴) (FIG. 5 (D)), corroborating possibilities of greater accessibility to extra-mitochondrial degradation mechanisms. A minimal inverse correlation was observed between half-life and protein abundance in both the heart (ρ=−0.46 and P<2.2×10⁻¹⁶) and the liver (ρ=−0.19, P=7.95×10⁻³) (FIG. 6), whereas no significant correlation was observed between turnover rate and protein molecular weight, isoelectric point, hydrophobicity (FIG. 6). Taken together, these data argue that protein kinetics, similar to abundance, is a selectable trait of the proteome subject to cellular regulations.

### Turnover of Multiprotein Complexes

As mentioned above, protein turnover rates within mitochondrial types are quite variable. The subunits of multiprotein complexes have been suggested to have coordinated turnover, but notable exceptions were also reported. In this study, subunits of well-defined protein complexes displayed variable kinetics, but particular members of intermediate subcomplexes may turn over together in a tighter fashion. For instance, in the respiratory chain complex I, assembly factors turned over considerably faster than the protein complex median. In the heart, NDUFAF2 and NDUFAF3 had k=0.053 and 0.078 d⁻¹, compared to the median complex I value of 0.036±0.007 d⁻¹. The assembly factor proteins are integral to complex I topogenesis but dissociate from the mature complex. On the contrary, the core subunits of the Q subcomplex (NDUFS2, NDUFS3, NDUFS7, and NDUFS8) turned over similarly (heart: k=0.039 d⁻¹, 0.036 d⁻¹, 0.042 d⁻¹, 0.039 d⁻¹). This suggests that subunits with faster turnover may be more frequently exposed to or have existed as free monomers due to assembly sequence or topology. Under this scenario, turnover rates are influenced by the stability of association with the final assembly, whereas in the synchronized complex model, all constitutive subunits would have similar turnover kinetics. The inventors further examined the data on the subunit NDUFA9, which has a relatively fast turnover among all subunits only in the liver (k=0.27 d⁻¹), but not in the heart (k=0.035 d⁻¹). In complex I biogenesis, the Q subcomplex first assembles before NDUFA9 associates with the mitochondrial-encoded ND1 to initiate the assembly of the next intermediate. ND1 has a considerably lower abundance in the liver than in the heart compared to other subunits, a scenario consistent with increased surplus NDUFA9 free subunits. Likewise, the NDUFA4 and NDUFS7 subunits have above-median turnover in both organs (NDUFA4: heart, k=0.047 d⁻¹, liver, k=0.30 d⁻¹; NDUFS7: heart, k=0.042 d⁻¹, liver, k=0.028 d⁻¹) and are incorporated only after stable intermediates are formed.

### Protein Turnover in Mitochondria

It is known that autophagy could degrade whole mitochondria when induced, and act as a synchronizing mechanism for protein kinetics inside the organelle. Indeed, the overall range of mitochondrial protein turnover rates observed here is much narrower than that reported for the cellular proteome. Nevertheless, the observation that individual mitochondrial protein turnover rates span at least an order of magnitude within an organ suggests individual mitochondria cannot be simplistically assumed to turn over only as single units. In theory, the assumption of steady-state protein abundance in inferring turnover from synthesis may be transiently offset by bouts of occasional mitophagy and remains valid over the labeling period. However, since the inventors measured protein turnover from isolated mitochondria, it was reasoned that given the observed variability in synthesis rates, at any moment each mitochondrion would contain some proteins that have been more recently synthesized than others. If mitophagy is predominant in the process of mitochondrial protein removal, then many mitochondria in the cell would be missing critical components. Since this circumstance is unlikely, a mechanism is necessary to allow mitochondria with new and old proteins to preserve homeostasis under mitophagy. Mitochondrial proteins may be synthesized in excess in the cytosol at variable rates before entering the mitochondria simultaneously. Alternatively, a sorting mechanism prior to autophagy would exist such that some protein species are preferentially recycled during fusion-fission cycles. Given evidence of either possibility remains scarce, the inventors posit that it is likely individual substrate proteolysis plays significant roles in mitochondrial dynamics. The asynchronous degradation of mitochondrial proteins is attested by the multiple protease complexes inside mitochondria. Under this model, measuring total organellar protein synthesis as a proxy for homeostasis would be inadequate to capture the details of mitochondrial protein turnover. These findings underscore the significance of obtaining a proteome dynamics map at individual protein resolution in uncovering signatures of protein quality control dysfunctions, such as in aging and metabolic perturbation studies.

In conclusion, demonstrated herein is the first mitochondrial proteome-wide study of in vive protein dynamics. The experimental platforms tailored to the analysis of changes in mass isotopomer distribution enabled the inventors to determine the turnover rates of 458 murine mitochondrial proteins, spanning over 2 orders of magnitude in half-life. Mitochondrial protein turnover displays both organ-specific differences and inter-protein heterogeneity, whereas subcellular fractionation ensured the protein kinetics were free from interference by cytosolic precursors. These kinetic data will help elucidate the mechanisms of mitochondrial homeostasis. This methodology has wide applications in the characterization of protein kinetics and temporal proteome changes in mammalian systems. The safety and economy of ²H₂O labeling also make it practical to measure human protein dynamics in clinical studies.

### Example 2

This example describes methodologies used to determine the turnover rates of human plasma proteins in six healthy human participants using the methods disclosed herein.

### Reagents

²H₂O (70% and 99.9% molar ratio purity) was obtained from Cambridge Isotope Laboratories. Other chemical reagents were acquired from Sigma-Aldrich unless otherwise specified. Milli-Q (Millipore) filtered water (18.2 MCI) was used for all sample preparation. HPLC-grade water (J.T.Baker) was used for LC solvent preparation.

### Stable Isotope Labeling in Human

Six healthy participants gave written informed consent. The participants were instructed to intake 4 boluses of 0.51-mL/kg sterile 70% molar ratio ²H₂O daily at 11:00 am, 2:00 pm, 6:00 pm, and 9:00 pm for the first 7 days; and 2 boluses of 0.74-mL/kg sterile 70% molar ratio ²H₂O daily at 11:00 am and 9:00 pm for the next 7 days. During the labeling period, participants were given daily general physical examinations and inquired for adherence to the labeling regimen. From day 0 to day 14, 2 mL of saliva samples and 3 mL of whole blood samples were collected daily at 12:00 noon for a total of 15 time points. The participants were monitored for 14 days to 6 months post-administration, and indicated no discomfort or side effects throughout the labeling and monitoring periods.

### Sample Preparation

The human whole blood sample was collected in lithium heparin tubes and separated into plasma and erythrocytes by centrifugation. 7 μL of plasma samples (approximately 500 μg) were depleted of the 14 top abundance proteins using Agilent Hu14 spin cartridges.

### GC-MS

Human plasma was centrifuged for 20 min at 14000 g at 4° C. For each sample, 20 μL of plasma was mixed with 2 μL of 10 N NaOH and 4 μL of 5% (v/v) acetone in acetonitrile. The standard curves were created by adding 1% to 20% molar ratio of ²H₂O at 1% intervals in 1× phosphate-buffered saline to acetone in place of the body fluid sample. The sample-acetone mixtures were incubated at ambient temperature overnight. Acetone was extracted by adding 500 μL of chloroform and 0.5 g of anhydrous sodium sulfate. 1 μL of the extracted solution analyzed on a GC mass spectrometer (Agilent 6890/5975) with a DB17-MS capillary column (Agilent, 30 m×0.25 mm×0.25 μm) at the UCLA Molecular Instrumentation Center. The column temperature gradient was as follows: 60° C. initial, 20° C.·min−1 increase to 100° C., 50° C.·min−1 increase to 220° C., 1 min hold. The mass spectrometer operated in the electron impact mode (70 eV) and selective ion monitoring at m/z 58 and 59 with 10 ms dwelling time.

### LC-MS/MS

Online low-PH reversed-phase LC was performed on all samples using a Thermo Easy-nLC 1000 nano-UPLC system on an EasySpray C18 column (PepMap, 3-μm particle, 100-Å pore; 75 μm×150 mm dimension) held at 50° C. Solvents for LC separation were as follows—solvent A: 0.1% formic acid, 2% acetonitrile; solvent B: 0.1% formic acid, 80% acetonitrile. The LC gradient was as follows—0-110 min: 0-40% B; 110-117 min: 40-80% B; 117-120 min: 80% B; 300 nL·min-1. 10 μL of each first-dimension RP fraction was injected onto the column through the integrated autosampler on the LC system.

Mass spectrometry was performed on an LTQ Orbitrap Elite mass spectrometer (Thermo Fisher Scientific) controlled by XCalibur version 2.1.0 coupled to the Easy-nLC 1000 system through a Thermo EasySpray interface. Each survey scan was analyzed in the orbitrap at 60,000 resolving power in profile mode, followed by data-dependent collision-induced dissociation MS2 scans on the top 15 ions in the ion trap. MS1 and MS2 target ion accumulation values are 1×104 and 1×106, respectively. Dynamic exclusion was set to 90 s. A lock mass of m/z 425.120025 was used for MS1.

### Data Analysis

The [.raw] raw spectrum files were converted to [.ms2] formats using Raw Xtractor (v.1.9.9.2) then searched using ProLuCID on the Integrated Proteomics Pipeline against a reverse-decoyed database (human: Uniprot Reference Proteome Reviewed, Feb. 9, 2013, 20,241 entries). Up to 3 variable methionine oxidations (+15.9949 Da) and static cysteine carbamidomethylation (+57.02146 Da) were allowed. Fully-tryptic, half-tryptic, and non-tryptic peptides within a 50 ppm mass windows surrounding the candidate precursor mass were searched. Protein identifications were filtered by DTASelect using 1% global peptide false discovery rate and two identified peptides per protein. False positive identifications were further controlled by requiring a peptide to be explicitly identified in at least 7 out of 15 time points in the human sample experiments. Normalized spectral abundance factors were extracted from the spectral counts in DTASelect.

The [.raw] Orbitrap elite spectrum files were converted to [.mzML] format using MSConvert. Data quantification was performed with ProTurn. ProTurn selected confidently identified peptides that are uniquely assigned to proteins and integrated the areas-under-curves of the peptide mass chromatographs based on MS2 scan numbers. In the human plasma samples, peptides explicitly identified in at least 7 out of 15 time points are accepted. The integrated mass isotopomer fractional abundance information was fitted using the Nelder-Mead method by ProTurn to optimize for k. Optimization results were independently verified by two data-fitting scripts written in R and MATLAB. The quality of the fitting was estimated as [1−(residual sum of squares)/total sum of squares)] (R²). Only peptide isotopomer time-series fitted by the model with R²≥0.9 were accepted. The error range of fitted k is measured by dk/dA0×σA where σA is the residual sum of square after optimization.

### Results

The participants were given regular, body-mass-adjusted boluses of sterile 70% ²H₂O for 14 days. Whole blood samples were taken daily. Because of the inherent biomass of the human body, ²H₂O enrichment levels do not plateau instantaneously in human labeling experiments. In the six participants, body water ²H₂O levels gradually approached the plateau levels of 1.8%, 2.2%, 2.1%, 1.6%, 1.0%, and 1.7% at the rate of 0.258 d⁻¹, 0.147 d⁻¹, 0.161 d⁻¹, 0.159 d⁻¹, 0.238 d⁻¹, and 0.176 d⁻¹ respectively. A first-order exponential decay function would not accurately model the isotopomer time-evolution under gradual ²H₂O enrichment. Therefore the inventors derived a nonlinear fitting model that accounts for both precursor and protein enrichment rates to explain isotopomer distributions. Briefly, the decreases in the relative abundance of the 0th peptide isotopomer (dA0/dt) due to 2H enrichment follow the first-order kinetics equation below (Equation 1):

dA₀/dt=k(A_{0,max}−A₀) (1)

Changes in peptide isotopomer fractional abundance are governed by A_{0,max}, which denotes the maximum amount of ²H label entering the protein at a particular time. A_{0,max} varies with the precursor enrichment level, p. it was reasoned that as an animal represents a well-mixed system of total water, ²H₂O enrichment would in turn approximate first-order kinetics (Equation 2):

A_{0,max}=α(1−p)^{N}=α(1−pₛₛ(1−e^{-k}^{ₚ}^{t}))^{N} (2)

Substituting Equation 2 into Equation 1 and solving the resulting differential equation for dA₀/dt yields a function of five parameters: the protein turnover rate k, the ²H₂O enrichment rate, kp, the plateau ²H₂O enrichment, pss, the natural abundance of A0, a, and the number of ²H labeling sites on the peptide, N. The values of kp and pss are measured from body water. A0, and N for each peptide can be readily calculated from the abundance of natural isotope and the number of accessible hydrogen atoms on individual amino acids. Fitting the function to the experimental A0 values at multiple time points therefore yields the value of k.

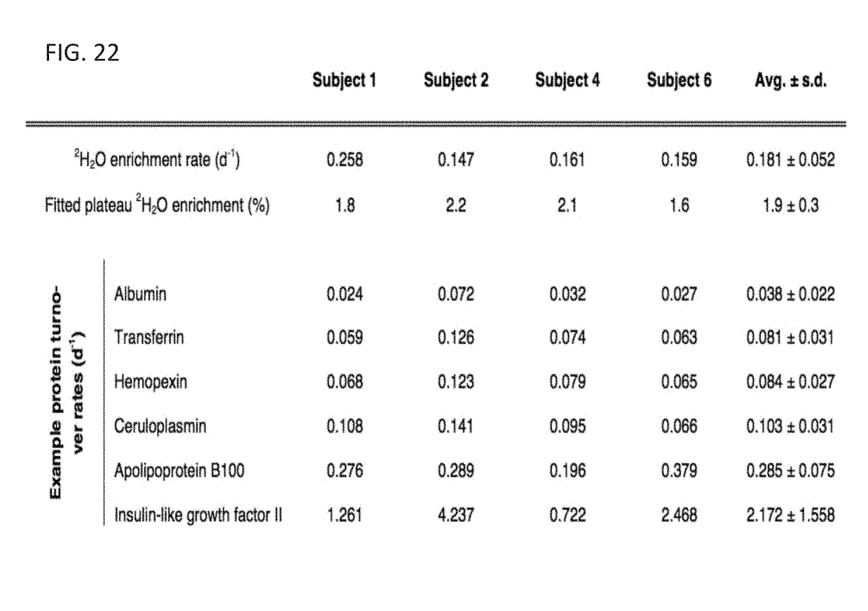

Using this model, the inventors deduced the turnover rates of 496 plasma proteins from the participants. The measured turnover rates span≈2 orders of magnitude. Data from the participants show excellent correlation (Pair-wise Spearman's ρ=0.82 to 0.93) and compare well with known turnover rates, whereas erythrocyte proteins did not turn over appreciably in the experimental period. In the plasma samples, the function modeled 32% to 47% consistently observed (≥7 of 15 time points) peptides with high precision (R²≥0.9); >50% of consistently identified proteins yielded confident kinetic information (R²≥0.9), indicating the nonlinear fitting model is compatible with large-scale inquiries. Examples of representative protein turnover rates as determined herein as shown in the Table given as FIG. 22

### Example 3

This example describes methodologies used to determine the turnover rates of young adult drosophila proteins using the method disclosed herein

The inventors designed a labeling strategy to examine protein turnover in adult drosophila and to study dynamics-related processes in aging, dietary restriction and proteolysis. Newly-enclosed adults were housed on agar-cornmeal-molasses-yeast media made in 12% ²H₂O for up to 21 days, and acquired body water ²H₂O enrichment of 10.9% at the rate of 0.94 d⁻¹. Mass spectrometry data from total cytosolic proteins harvested at 8 time points revealed that a number of drosophila peptides possessed fewer ²H₂O-labeling sites than predicted, possibly due to differences in amino acid metabolism between drosophila and mammals. The inventors therefore performed multivariate optimization for best-fit values of both k and N, which deduced the turnover rates of 491 peptides belonging to 247 proteins with high confidence (R²≥0.9) (compared to 181 proteins when optimizing for k alone). The median measured protein half-life in the experiment was 3.6 days. Functional clusters showed different protein kinetics, with glycolysis proteins having 3.9 times longer median half-life than proteasome subunits (9.5 days vs. 2.4 days).

### Method

Wild-type Oregon-R-c drosophila were acquired from Bloomington Drosophila Stock Center and propagated on standard media prepared by the UCLA Drosophila Media Facility. Labeled media were made by dissolving 12 g of Drosophila agar type II (Diamed) in 12% molar ratio of ²H₂O in 1 L of water with heating to 85° C., then mixing in 29 g of yeast (Red Star), 71 g of cornmeal (Quaker), and 92 g of molasses (Grandma's brand), 16 mL of 10% methylparaben in ethanol, and 10 mL of 50% propionic acid. Newly-enclosed adults were housed in 300 mL plastic fly bottles containing 50 mL of the set media on the bottom, at a density of ˜450 flies per bottle, at ambient temperature for up to 21 days. Drosophila were transferred to fresh bottles every 3 days during labeling and harvested at 8 time points (0, 1, 2, 4, 7, 10, 14, 21 d). Drosophila adults (˜200 mg) were homogenized with a pestle and mortar with 4 mL of the extraction buffer at 4° C. The homogenates were filtered by a cell strainer (BD Falcon) then centrifuged at 800 g at 4° C. for 7 min. The supernatant was centrifuged again at 4000 g at 4° C. for 30 min and collected.

The concentrations of the extracted proteins were measured by a bicinchoninic acid assay (Thermo Pierce). Mouse protein samples were digested in-solution. 200 μg proteins were heated at 80° C. with 0.2% (w/v) Rapigest (Waters) for 5 min, then heated at 70° C. with 3 mM dithiothreitol for 5 min, followed by alkylation with 9 mM iodoacetamide in the dark at ambient temperature. Proteins were digested with 50:1 sequencing grade trypsin (Promega) for 16 h at 37° C., then acidified with 1% trifluoroacetic acid (Thermo Pierce). Depleted human plasma samples were digested on-filter using 10,000 Da filters (Pall Life Sciences). Sample buffer was exchanged on-filter with 100 mM ammonium bicarbonate. The samples were then heated on-filter at 70° C. with 3 mM dithiothreitol for 5 min, followed by alkylation with 9 mM iodoacetamide in the dark at ambient temperature. Proteins were digested with 50:1 sequencing grade trypsin (Promega) for 16 h at 37° C. Drosophila protein samples (200 μg) were heated at 70° C. in Laemmli Sample Buffer for 5 min, then separated on a 12% Tris-glycine acrylamide gel with 6% stacking gel at 80 V at ambient temperature for 19 h. The gel was stained with Coomassie and cut into 21 regular fractions. Each fraction was digested in-gel with 30:1 sequencing-grade trypsin (Promega) following reduction and alkylation by dithiothreitol and iodoacetamide. Extracted peptides were reconstituted in 0.1% formic acid, 2% acetonitrile and injected for second-dimension LC. Drosophila body fluid was extracted from 450 mg of drosophila adult at each time point, through homogenization with a Teflon pestle and filtration with a C18 cartridge (Thermo Pierce). For each sample, 20 μL of plasma or body fluid was mixed with 2 μL of 10 N NaOH and 4 μL of 5% (v/v) acetone in acetonitrile. The standard curves were created by adding 1% to 20% molar ratio of ²H₂O at 1% intervals in 1× phosphate-buffered saline to acetone in place of the body fluid sample. The sample-acetone mixtures were incubated at ambient temperature overnight. Acetone was extracted by adding 500 μL of chloroform and 0.5 g of anhydrous sodium sulfate. 1 μL of the extracted solution analyzed on a GC mass spectrometer (Agilent 6890/5975) with a DB17-MS capillary column (Agilent, 30 m×0.25 mm×0.25 μm) at the UCLA Molecular Instrumentation Center. The column temperature gradient was as follows: 60° C. initial, 20° C.·min⁻¹ increase to 100° C., 50° C.·min⁻¹ increase to 220° C., 1 min hold. The mass spectrometer operated in the electron impact mode (70 eV) and selective ion monitoring at m/z 58 and 59 with 10 ms dwelling time.

LC-MS and Data Analysis are conducted in identical manner as described in Example 2.

### Example 4

This example describes methodologies used to compare the turnover rates of mammalian cardiac proteins with or without stimuli using the methods disclosed herein.

To illustrate sensitivity in detecting turnover changes, proteome kinetics were compared in the hearts of mice being administered 15 mg·kg⁻¹·d⁻¹ isoproterenol for 14 d to induce cardiac hypertrophy. Using this model, the inventors deduced the turnover rates of 2,964 cardiac proteins from the animals. The measured turnover rates spanned≈2 orders of magnitude. The results showed that isoproterenol stimulation was led to widespread acceleration in protein turnover in the mouse heart, with the average turnover rates being measured about 1.23-fold higher than in the normal heart. Despite that the cardiac proteome does not remain constant during remodeling, the nonlinear kinetic method disclosed herein calculated turnover rates precisely and represented the majority of protein turnover behaviors. On the whole, proteins with significant changes after isoproterenol stimulation belong to at least 35 biological processes that present promising targets for further studies. Following isoproterenol treatment, the turnover rates of several proteins previously implicated in cardiac remodeling and heart failure, including collagen XV, annexin V, and endonuclease G are specifically increased (76^{th} to 98^{th} percentile, but their overall abundance did not change noticeably when evaluated by label-free quantification techniques. This indicates that kinetics measurements could detect stimuli-induced responses in a physiologically relevant setting.

### Method

Labeling was initiated by two i.p. injections of 500 μL 99.9% ²H₂O-saline spaced 4 h apart. Mice were then given free access to 8% ²H₂O in the drinking water supply. Groups of 3 mice each were euthanized on day 0, 1, 2, 3, 5, 7, 10, 14 following the initiation of labeling (first ²H₂O i.p. injection) at 12:00 noon for sample collection. A separate set of male Hsd:ICR mice were surgically implanted with micro-osmotic pumps (Alzet) calibrated to deliver 15 mg·kg⁻¹·d⁻¹ isoproterenol. Labeling was initiated simultaneously with the micro-osmotic pump as above. Mouse hearts were excised and homogenized by a 7-mL Dounce homogenizer (Pyrex) (20 strokes) in an extraction buffer (250 mM sucrose, 10 mM HEPES, 10 mM Tris, 1 mM EGTA, 10 mM dithiothreitol, protease and phosphatase inhibitors (Pierce Halt), pH 7.4) at 4° C., then centrifuged (800 g, 4° C., 7 min). The pellet was collected as the total debris fraction. The supernatant was centrifuged (4,000 g, 4° C., 30 min) and collected as the organelle-depleted cytosolic fraction. The pellet was washed, then overlaid on a 19%/30%/60% discrete Percoll gradient, and sedimented by ultracentrifugation (12,000 g, 4° C., 10 min). Purified mitochondria were collected from the 30%/60% interface layer and washed twice. Protein concentrations were measured by bicinchoninic acid assays (Thermo Pierce). Prior to online low-pH reversed-phase LC, mouse sample peptides were first separated on a Finnigan Surveyor LC system using a Phenomenex C18 column (Jupiter Proteo C12, 4 μm particle, 90 Å pore, 100 mm×1 mm dimension). Solvents were as follows—Solvent A: 20 mM ammonium formate, pH 10; solvent B, 20 mM ammonium formate, 90% acetonitrile). The gradient was as follows: 0-2 min, 0-5% B; 3-32 min, 5-35% B; 32-37 min, 80% B; 50 μL μL·min⁻¹. 50 μg of tryptic peptides were injected with a syringe into a manual 6-port/2-position switch valve. Fractions were collected every 2 minutes. The fractions 9 to 20 were lyophilized and re-dissolved in 20 μL 0.5% formic acid prior to low-pH reversed-phase separation.

LC-MS and data analysis was carried out in identical manners as in Example 2.

### Example 5

This example describes methodologies used to identify biomarkers of disease by measuring the turnover rates of biomolecules, such as proteins, in body fluids (e.g., blood or saliva) after pathological stimuli using the methods disclosed herein.

The inventors designed a labeling strategy to identify biomarkers from an accessible tissue that reflect the development of disease in the heart following a pathological stimulus. The inventors were able to determine the turnover rates of 295 and 238 proteins from the plasma of mouse with or without the stimulus, respectively. It was found that contrary to cardiac proteins, the majority of plasma proteins displayed decreased turnover rates following the stimulus. Particular proteins nevertheless displayed elevated turnover rates, including parvalbumin, suggesting they may be associated with stimulus responses.

### Methods

Label initiation and isoproterenol stimulus were carried out in identical manners as in Example 4. Mouse plasma samples were fractionated from blood centrifugate and digested in-solution; 200 μg proteins were heated at 80° C. with 0.2% (w/v) Rapigest (Waters) for 5 min, then heated at 70° C. with 3 mM dithiothreitol for 5 min, followed by alkylation with 9 mM iodoacetamide in the dark at ambient temperature. Proteins were digested with 50:1 sequencing grade trypsin (Promega) for 16 h at 37° C., then acidified with 1% trifluoroacetic acid (Thermo Pierce). Depleted human plasma samples were digested on-filter using 10,000 Da polyethersulfone filters (Nanosep; Pall Life Sciences). Sample buffer was exchanged on-filter with 100 mM ammonium bicarbonate. The samples were then heated at 70° C. with 3 mM dithiothreitol for 5 min, followed by alkylation with 9 mM iodoacetamide in the dark at ambient temperature. Proteins were digested with 50:1 sequencing grade trypsin (Promega) on-filter for 16 h at 37° C. LC-MS and data analysis was carried out in identical manners as in Example 2.

### Example 6

This example describes methodologies used to compare the turnover rates of mammalian cardiac proteins during the recovery stage following the withdrawal of a pathological stimulus using the methods disclosed herein.

A set of male Hsd:ICR mice were surgically implanted with micro-osmotic pumps (Alzet) calibrated to deliver 15 mg·kg⁻¹·d⁻¹ isoproterenol for 14 days. After day 14 of pump implantation, the isoproterenol source inside the micro-osmotic pump became depleted and the mice began to gradually recover from the stimulus. Labeling started 14 days after the micro-osmotic pump was installed, initiated by two i.p. injections of 500 μL 99.9% ²H₂O-saline spaced 4 h apart. Mice were then given free access to 8% ²H₂O in the drinking water supply. Groups of 3 mice each were euthanized on day 14, 15, 16, 17, 19, 21, 24, 28 following the installation of the micro-osmotic pumps at 12:00 noon for sample collection.

LC-MS and data analysis was carried out in identical manners as in Example 2. The inventors deduced the turnover rates of 2,034 proteins from the cardiac cytosol following the withdrawal of isoproterenol stimulus. Based on the directions of kinetic changes following isoproterenol stimulation and subsequent withdrawal, the kinetic behaviors of proteins were categorized into four types. In the first type, reverse cardiac remodeling reversed the elevated turnover observed during isoproterenol stimulus. This group encompassed most proteins, but was most prominently enriched for ribosome subunits (Fisher P≤8.6×10⁻⁷). The second type of protein behaviors displayed elevated turnover in isoproterenol stimulation that sustained following withdrawal. This group was functionally distinguished from the first by its significant enrichment of MAPK signaling proteins (Fisher P≤6.3×10⁻⁵). Relatively few proteins showed decreased turnover throughout remodeling and reverse remodeling, a group suggestively enriched for proteolysis pathway proteins (Fisher P≤9.2×10⁻⁴). The data demonstrate that the methods disclosed herein can be used to trace the recovery of a biological system from a pathological perturbation.

### Example 7

This example describes methodologies used to determine turnover rates of human plasma proteins from samples taken at a single time point in two healthy human participants using the methods disclosed herein.

The inventors designed a labeling strategy to discern protein turnover rates from just a single blood sample from a human subject. Extant labeling methods would require repeated sample biopsies, which presents unnecessary distress and is impractical in many clinical settings. In the nonlinear modeling method disclosed herein, the initial and final isotopomer abundances of a peptide in the MS (i.e., the unlabeled and fully turned over protein, respectively) can be precisely defined by the peptide sequence and ²H₂O enrichment in the body water. One or more data points acquired from a single time point in between could therefore sufficiently demarcate the trajectory of the kinetic curve. To demonstrate the feasibility of deducing protein kinetics deduction from a single, non-time-course measurement, the inventors procured human plasma samples from each of three individual time points (day 4, 8, and 12) following the beginning of labeling in two subjects.

Human subject labeling, LC-MS and data analysis were carried out in identical manners as in Example 2. The results showed that although the day 4 samples presented more variations—possibly due to limited label incorporation, both the day-8 and the day-12 single-point measurements were highly consistent with multi-point time-course data (correlation coefficient=0.81 to 0.93).

Because cardiac proteins have limited surgical accessibility and are typically only available during cardiac transplant or ventricular assist device implantation, the described method opens opportunities for kinetic investigations of the human heart, among other invasive tissue samples. Heart transplant recipients undergo regular scheduled cardiac biopsies within the first year following transplantation to diagnose for allograft rejection. The schedule of the biopsy permits the patient to be labeled up to two weeks prior in identical manners as described in Example 2. The operation procures an endomyocardial biopsy 2 mm×2 mm×2 mm in dimension from the intraventricular septum using a bioptome. From that amount of human heart, the inventors were able to extract 400 μg of total cardiac proteins, and identified 863 total human cardiac protein species from 2 μg of proteins, demonstrating protein turnover rate from a healthy adult heart to be deduced from one time point using the methods disclosed herein.

### Example 8

This example describes methodologies used to compare the turnover rates of mammalian proteins from diverse genetic backgrounds using the methods disclosed herein.

The inventors designed a method to study the genetic contribution to disease susceptibility by measuring protein turnover rates in a mouse inbred strain known to be resistant to heart failure after isoproterenol stimulus, and one known to be susceptible. 8 FVB/NJ mice and 8 BALB/cJ mice were labeled by two i.p. injections of 500 μL 99.9% ²H₂O-saline spaced 4 h apart. Mice were then given free access to 8% ²H₂O in the drinking water supply for up to 5 days. Two mice from each strain was euthanized on day 0, 1, 3, 5 at 12:00 noon following the initiation of labeling, and cardiac proteins were procured in identical manners as described in Example 4. LC-MS and data analysis were carried out in identical manners as in Example 2.

The results suggested that at the basal level, the two genetic backgrounds of mice presented with different protein turnover rates. For example, the heat-shock 70 kDa protein (HSPA9) turned over at 1.46-fold rate in the disease-resistant FVB/NJ mice, but another protein, myosin regulatory light chain 7 (My17) was 0.52-fold in turnover rate. These data demonstrate the methods disclosed herein could be used to discern previously unknown manifestations of genetic differences and their associations with disease onset and progression.

### Example 9

This example describes methodologies used to compare the turnover rates of total proteins from neonatal rat ventricular myocyte (NRVM) cultures with or without genetic manipulation using the methods disclosed herein. NRVM cultures were used as an example to demonstrate the feasibility of the methods. The methods are applicable to all cell types, including cells from mice, human, primary cell cultures, transformed cell lines, induced pluripotent stem cells, embryonic stem cells, or induced differentiated cells.

The inventors designed a method to study the effect of a genetic perturbation on protein expression and synthesis rates in an in vitro cell culture system. NRVM were harvested, plated and cultured at the density of 3 million cells per plate. The cells were treated with a pathological stimulus, 50 μM phenylephrine, for 24 hours, then treated with a small interfering RNA (siRNA) to silent the expression of a gene product that modifies the response to the stimulus. To initiate labeling, the siRNA treated and control (off-target siRNA) cells were switched over to a cell culture medium (DMEM) that was enriched with 5% ²H₂O. The cells were harvested by scraping off the petri dish at day 1, 2, and 4 after the initiation of labeling. The inventors extracted 250 Mg of total myocyte proteins from each plate using 1×RIPA buffer (30 min incubation on ice) and sonication, then digested and fractionated the samples in identical manners as described in Example 4. Labeling of cellular water was assumed to be 5% since the medium was in excess to the cells. Nevertheless, the enriched medium was collected at every medium change for GC analysis. LC-MS and data analysis was carried out in identical manners as in Example 2. These experiments demonstrate the disclosed methods can be used to study in vitro cell system and the effects of genetic manipulation on protein turnover.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1-15. (canceled)

16. A computer-implemented method for determining the turnover rate of one or more biomolecules in a subject, the method comprising:
receiving, by one or more computing devices, mass spectra data from samples collected from a subject at one or more time points, wherein the one or more biomolecules in the subject have been labeled with ²H;
receiving, by the one or more computing devices, biomolecule identification data;
parsing, by the one or more computing devices, the mass spectra data and the biomolecule identification data;
assigning, by the one or more computing devices, mass spectral data to biomolecular identification data to identify peaks in the mass spectral data;
integrating, by the one or more computing devices, peaks in the mass spectral data to determine fractional abundance of one or more isotopomers of ²H labeled biomolecules in the samples;
receiving, by the one or more computing devices, enrichment rate and level data; and
fitting, by the one or more computing devices, the fractional abundance of the one or more isotopomers of ²H labeled biomolecules in the samples to an equation describing labeled biomolecule turn over to determine the molecular turnover rates of biomolecules in the subject.

17. The method of claim 16, further comprising providing, by the one or more computing devices, output of the molecular turnover rates of biomolecules in the subject.

18. The method of claim 16, further comprising filtering, by the one or more computing devices, the mass spectral data to determine the quantifiability of the mass spectral data.

19. The method of claim 16, wherein determining the biomolecule turnover rates of the one or more labeled biomolecules based on the fractional abundance of the one or more isotopomers comprises applying a unified kinetic model that predicts biomolecule labeling behavior under both constant and time-variable precursor stable isotope enrichment.

20. The method of claim 19, wherein the kinetic model comprises a first-order kinetic model of the precursor enrichment in the biological sample to predict the precursor enrichment level in a time-variable enrichment.

21. The method of claim 16, wherein determining the biomolecule turnover rates of the one or more labeled biomolecules based on the fractional abundance of the one or more isotopomers further comprises application of a governing equation of both precursor enrichment rate and protein enrichment rate, and the use of nonlinear fitting optimization methods to directly calculate turnover rate from mass spectra.

22. The method of claim 16, wherein determining the biomolecule turnover rates of the one or more labeled biomolecules based on the fractional abundance of the one or more isotopomers further comprises modeling the number of labeling sites in the biological samples, the natural fractional abundance of the one or more isotopomers, and its plateau fractional abundance during and after labeling.

23. The method of claim 16, wherein the biomolecule is a protein, nucleic acid, lipid, glycan, carbohydrate, or small molecule metabolite.

24. (canceled)

25. (canceled)

26. A system for determining the turnover rate of a biomolecule in a subject, the system comprising:
a storage device; and
a processor communicatively coupled to the storage device and configured to execute application code instructions that are stored in the storage device to cause the system to:
receive mass spectra data from samples collected from a subject at one or more time points, wherein biomolecules in the subject have been labeled with ²H;
receive biomolecule identification data;
parse the mass spectra data and the biomolecule identification data;
assign mass spectral data to biomolecular identification data to identify peaks in the mass spectral data;
integrate peaks in the mass spectral data to determine fractional abundance of one or more isotopomers of ²H labeled biomolecules in the samples;
receive enrichment rate and level data; and
fit the fractional abundance of the one or more isotopomers of ²H labeled biomolecules in the samples to an equation describing labeled biomolecule turnover to determine the molecular turnover rates of biomolecules in the subject.

27. (canceled)

28. The system of claim 26, wherein the processor is configured to execute further application code instructions that are stored in the storage device and that cause the system to:
filter the mass spectral data to determine the quantifiability of the mass spectral data.

29. The system of claim 26, wherein determining the biomolecule turnover rates of the one or more labeled biomolecules based on the fractional abundance of the one or more isotopomers comprises application of a unified kinetic model that predicts biomolecule labeling behavior under both constant and time-variable precursor stable isotope enrichment.

30. The system of claim 29, wherein the kinetic model comprises a first-order kinetic model of the precursor enrichment in the biological sample to predict the precursor enrichment level in a time-variable enrichment.

31. The system of claim 26, wherein determining the biomolecule turnover rates of the one or more labeled biomolecules based on the fractional abundance of the one or more isotopomers further comprises application of a governing equation of both precursor enrichment rate and protein enrichment rate, and the use of nonlinear fitting optimization methods to directly calculate turnover rate from mass spectra.

32. The system of claim 26, wherein determining the biomolecule turnover rates of the one or more labeled biomolecules based on the fractional abundance of the one or more isotopomers further comprises modeling the number of labeling sites in the biological samples, the natural fractional abundance of the one or more isotopomers, and its plateau fractional abundance during and after labeling.

33. A computer program product comprising:
a non-transitory computer-readable storage device having computer-readable program instructions embodied thereon that when executed by a computer cause the computer to perform a method for determining the turnover rates of biomolecules in a subject, the computer-executable program instructions comprising:
computer-executable program instructions to receive mass spectra data from samples collected from a subject at one or more time points, wherein biomolecules in the subject have been labeled with ²H;
computer-executable program instructions to receive biomolecule identification data;
computer-executable program instructions to parse the mass spectra data and the biomolecule identification data;
computer-executable program instructions to assign mass spectral data to biomolecular identification data to identify peaks in the mass spectral data;
computer-executable program instructions to integrate peaks in the mass spectral data to determine fractional abundance of one or more isotopomers of ²H labeled biomolecules in the samples;
computer-executable program instructions to receive enrichment rate and level data; and
computer-executable program instructions to fit the fractional abundance of the one or more isotopomers of ²H labeled biomolecules in the samples to a equation describing labeled biomolecule turnover to determine the molecular turnover rates of biomolecules in the subject.

34. (canceled)

35. The computer-executable program product of claim 33, further comprising computer-executable program instructions to filter the mass spectral data to determine the quantifiability of the mass spectral data.

36. The computer-executable program product of claim 33, wherein determining the biomolecule turnover rates of the one or more labeled biomolecules based on the fractional abundance of the one or more isotopomers comprises a unified kinetic model that predicts biomolecule labeling behavior under both constant and time-variable precursor stable isotope enrichment.

37. The computer-executable program product of claim 36, wherein the kinetic model comprises a first-order kinetic model of the precursor enrichment in the biological sample to predict the precursor enrichment level in a time-variable enrichment.

38. The computer-executable program product of claim 33, wherein determining the biomolecule turnover rates of the one or more labeled biomolecules based on the fractional abundance of the one or more isotopomers further comprises a governing equation of both precursor enrichment rate and protein enrichment rate, and the use of nonlinear fitting optimization methods to directly calculate turnover rate from mass spectra.

39. The computer-executable program product of claim 33, wherein determining the biomolecule turnover rates of the one or more labeled biomolecules based on the fractional abundance of the one or more isotopomers further comprises modeling the number of labeling sites in the biological samples, the natural fractional abundance of the one or more isotopomers, and its plateau fractional abundance during and after labeling.
